(19) United States
(12) Patent Application Publication
(10) Pub. No.: US 2011/0275638 A1
(43) Pub. Date: Nov. 10, 2011

(54) PIPERAZINE DERIVATIVES AND THEIR USE AS LEPTIN RECEPTOR MODULATORS

(76) Inventors: Emma Chapman, Cambridge (GB); Michael Higginbottom, Cambridge (GB); Anne Viet-Anh Horgan (nee Nguyen), London (GB); Iain Simpson, Cambridge (GB)

(21) Appl. No.: 12/996,623

(22) PCT Filed: Jun. 4, 2009
(86) PCT No.: PCT/EP09/56897

§ 371 Date: Jul. 26, 2011

(30) Foreign Application Priority Data
Jun. 4, 2008 (SE) 0801321-1

(51) Int. Cl. A61K 31/5377; A61K 31/495; C07D 213/55; A61K 31/44; C07D 405/12; A61K 31/443; C07D 413/12; A61P 3/04; A61P 3/10; A61P 3/00; A61P 9/12; A61P 15/08; A61P 17/00; A61P 27/02; A61P 37/00; A61P 17/02; A61P 9/10; C07D 241/04

(52) U.S. Cl. 514/237.2; 544/400; 514/252.12; 546/335; 514/357; 546/283.4; 514/336; 544/131

(57) ABSTRACT

The present invention relates to new compounds of formula (I), to pharmaceutical compositions comprising these compounds and to the use of these compounds as leptin receptor modulator mimetics in the preparation of medicaments against conditions associated with weight gain, type 2 diabetes and dyslipidemias.

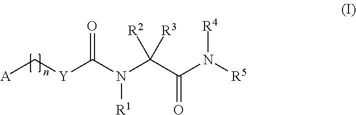

### FIELD OF THE INVENTION

The present application relates to new pyridine and piperazine derivatives, to pharmaceutical compositions comprising these compounds and to the use of these compounds as leptin receptor modulator mimetics in the preparation of medicaments against conditions associated with weight gain, type 2 diabetes and dyslipidemias.

### BACKGROUND ART

The prevalence of obesity is increasing in the industrialized world. Typically, the first line of treatment is to offer diet and life style advice to patients, such as reducing the fat content of their diet and increasing their physical activity. However, some patients may also need to undergo drug therapy to maintain the beneficial results obtained from adapting the aforementioned diet and lifestyle changes.

Leptin is a hormone synthesized in fat cells that is believed to act in the hypothalamus to reduce food intake and body weight (see, e.g., Bryson, J. M. (2000) Diabetes, Obesity and Metabolism 2: 83-89).

It has been shown that in obese humans the ratio of leptin in the cerebrospinal fluid to that of circulating leptin is decreased (Koistinen et al., (1998) Eur. J. Clin. Invest. 28: 894-897). This suggests that the capacity for leptin transport into the brain is deficient in the obese state. Indeed, in animal models of obesity (NZO mouse and Koletsky rat), defects in leptin transport have been shown to result in reduced brain leptin content (Kastin, A. J. (1999) Peptides 20: 1449-1453; Banks, W. A. et al., (2002) Brain Res. 950: 130-136). In studies involving dietary-induced obese rodents (a rodent model that is believed to more closely resemble human obesity, see, e.g., Van Heek et al. (1997) J. Clin. Invest. 99: 385-390), excess leptin administered peripherally was shown to be ineffective in reducing food intake and body weight, whereas leptin injected directly into the brain was effective in reducing food intake and body weight. It has also been shown that in obese humans with excess circulating leptin, the signaling system became desensitized to the continual stimulation of the leptin receptors (Mantzoros, C. S. (1999) Ann. Intern. Med. 130: 671-680).

Amgen has conducted clinical trials with recombinant methionyl human leptin. The results from these trials were mixed, as even in the presence of high plasma concentrations of leptin weight loss was variable, and the average weight reduction in the cohort of patients tested relatively small (Obesity Strategic Perspective, Datamonitor, 2001).

Several attempts at finding active fragments have been reported in the literature since the discovery of the leptin gene coding sequence. An example is by Samson et al. (1996) Endocrinol. 137: 5182-5185 which describes an active fragment at the N-terminal (22 to 56). This sequence was shown to reduce food intake when injected ICV whereas a sequence taken at the C-terminal was shown not to have any effect. Leptin fragments are also disclosed in International Patent Application WO 97/46585.

Other reports looking at the C-terminus part of the sequence reported a possible stimulation of luteinising hormone production by a 116-130 fragment (Gonzalez et al., (1999) Neuroendocrinology 70:213-220) and an effect on GH production following GHRH administration (fragment 126-140) (Hanew (2003) Eur. J. Endocrin. 149: 407-412).

Leptin has recently been associated with inflammation. It has been reported that circulating leptin levels rise during bacterial infection and in inflammation (see Otero, M et al. (2005) FEBS Lett. 579: 295-301 and references therein). Leptin can also act to increase inflammation by enhancing the release of pro-inflammatory cytokines TNF and IL-6 from inflammatory cells (Zarkesh-Esfahani, H. et al. (2001) J. Immunol. 167: 4593-4599). These agents in turn can contribute to the insulin resistance commonly seen in obese patients by reducing the efficacy of insulin receptor signaling (Lyon, C. J. et al. (2003) Endocrinol. 44: 2195-2200). Continuous low grade inflammation is believed to be associated with obesity (in the presence and absence of insulin resistance and Type II diabetes) (Browning et al. (2004) Metabolism 53: 899-903, Inflammatory markers elevated in blood of obese women; Mangge et al. (2004) Exp. Clin. Endocrinol. Diabetes 112: 378-382, Juvenile obesity correlates with serum inflammatory marker C-reactive protein; Maachi et al. (2004) Int. J. Obes. Relat. Metab. Disord. 28: 993-997, Systemic low grade inflammation in obese people). Leptin has also been implicated in the process of atherogenesis, by promoting lipid uptake into macrophages and endothelial dysfunction, thus promoting the formation of atherosclerotic plaques (see Lyon, C. J. et al. (2003) Endocrinol. 144: 2195-2200).

Leptin has also been shown to promote the formation of new blood vessels (angiogenesis) a process implicated in the growth of adipose tissue (Bouloumie A, et al. (1998) Circ. Res. 83: 1059-1066). Angiogenesis has also been implicated in diabetic retinopathy (Suganami, E. et al. (2004) Diabetes. 53: 2443-2448).

Angiogenesis is also believed to be involved with the growth of new blood vessels that feed abnormal tumour cells. Elevated leptin levels have been associated with a number of cancers, in particular breast, prostate and gastrointestinal cancers in humans (Somasundar P. et al. (2004) J. Surg. Res. 116: 337-349).

Leptin receptor agonists may also be used in the manufacture of a medicament to promote wound healing (Gorden, P. and Gavrilova, O. (2003) Current Opinion in Pharmacology 3: 655-659).

Further, it has been shown that elevating leptin signaling in the brain may represent an approach for the treatment of depressive disorders (Lu, Xin-Yun et al. (2006) PNAS 103: 1593-1598).

### DISCLOSURE OF THE INVENTION

It has surprisingly been found that compounds of formula (I) are effective in reducing body weight and food intake in rodents. While not wishing to be bound by theory, it is proposed that the compounds of formula I modulate the leptin receptor signaling pathway.

In some embodiments, compounds with leptin receptor agonistic like properties can be useful for the treatment of disorders relating to leptin signaling, as well as conditions associated with weight gain, such as obesity. The inventors hypothesized that small molecule CNS penetrant leptin mimetics would be able to by-pass the limiting uptake system into the brain. Further, assuming that this situation mirrors the human obese condition, the inventors believe that a CNS-penetrant leptinoid with a relatively long duration of action would make an effective therapy for the obese state and its attendant complications, in particular (but not limited to) diabetes.

In other embodiments, compounds with leptin receptor antagonistic like properties could be useful for the treatment of inflammation, atherosclerosis, diabetic retinopathy and nephropathy.

In a first aspect, the disclosure relates to a compound of formula (I),

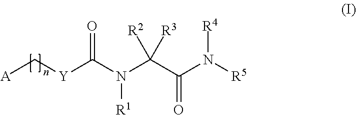

or a pharmaceutically acceptable salt, solvate, hydrate, geometrical isomer, tautomer, optical isomer or N-oxide thereof, wherein:

A is selected from pyridinyl and piperazinyl, each of which is optionally substituted with one or more C₁₋₄-alkyl groups;

Y is selected from O, N(R⁶) and CH₂;

R¹ is selected from hydrogen and C₁₋₄-alkyl;

R² is selected from hydrogen and C₁₋₄-alkyl;

R³ is selected from C₁₋₄-alkyl, hydroxy-C₁₋₄-alkyl and phenyl-C₁₋₄-alkyl, wherein phenyl is optionally substituted with one or more substituents independently selected from halogen, hydroxy, cyano, CF₃, C₁₋₄-alkyl and C₁₋₄-alkoxy;

R⁴ is selected from hydrogen and C₁₋₄-alkyl;

R⁵ is selected from C₁₋₆-alkyl (optionally substituted with one or more substituents independently selected from oxo and fluoro), phenyl-C₁₋₆-alkyl (wherein phenyl is optionally substituted with one or more substituents independently selected from halogen, hydroxy, cyano, CF₃, C₁₋₆-alkyl and C₁₋₆-alkoxy) and heterocyclyl-C₁₋₆-alkyl; or

R⁴ and R⁵, together with the nitrogen atom to which they are bound, form a saturated heterocyclic ring which is optionally substituted with one or more C₁₋₄-alkyl groups;

R⁶ is selected from hydrogen and C₁₋₄-alkyl; and

n is 1, 2 or 3;

with the proviso that the compound is not selected from:
N,3-dimethyl-2-[[[methyl(2-pyridinylmethyl)amino]carbonyl]amino]butanamide; and
N-[(1S)-1-[[[(1S)-1-(1,3-dioxolan-2-yl)-3-methylbutyl]amino]carbonyl]-2-methyl-propyl]-3-pyridinepropanamide.

In a preferred embodiment, Y is O.

R¹ is preferably hydrogen.

R² is preferably hydrogen or methyl.

R³ is preferably methyl, hydroxymethyl, benzyl, p-hydroxybenzyl or (p-hydroxyphenyl)-ethyl.

R⁴ is preferably hydrogen or methyl.

R⁵ is preferably methyl, isopropyl, 3-methylbutyl, 2,2-difluoroethyl, 3,3-dimethyl-2-oxobutyl, benzyl, 1-phenylethyl, 2-phenylethyl or tetrahydrofuran-2-ylmethyl; or

when R⁴ and R⁵, together with the nitrogen atom to which they are bound, form a saturated heterocyclic ring, said ring is preferably morpholine or 2,6-dimethylmorpholine.

n is preferably 1 or 2.

Specific preferred compounds according to the disclosure are those selected from the group consisting of:
2-piperazin-1-ylethyl{(1S)-1-(4-hydroxybenzyl)-2-[methyl(3-methylbutyl)amino]-2-oxoethyl}carbamate;
2-piperazin-1-ylethyl[(1S)-2-[benzyl(methyl)amino]-1-(4-hydroxybenzyl)-2-oxoethyl]-carbamate;
2-piperazin-1-ylethyl{(1S)-1-(4-hydroxybenzyl)-2-[methyl(2-phenylethyl)amino]-2-oxoethyl}carbamate;
2-piperazin-1-ylethyl{(1S)-1-(4-hydroxybenzyl)-1-methyl-2-[(3-methylbutyl)amino]-2-oxoethyl}carbamate;
pyridin-4-ylmethyl[(1S)-2-[benzyl(methyl)amino]-1-(4-hydroxybenzyl)-2-oxoethyl]-carbamate;
pyridin-4-ylmethyl{(1S)-1-(4-hydroxybenzyl)-2-[methyl(2-phenylethyl)amino]-2-oxo-ethyl}carbamate;
pyridin-4-ylmethyl{(1S)-1-(4-hydroxybenzyl)-2-[methyl(3-methylbutypamino]-2-oxo-ethyl}carbamate;
pyridin-4-ylmethyl((1S)-3-(4-hydroxyphenyl)-1-{[methyl(2-phenylethyl)amino]-carbonyl}propyl)carbamate;
pyridin-4-ylmethyl{(1S)-1-(hydroxymethyl)-2-[methyl(3-methylbutypamino]-2-oxo-ethyl}carbamate;
pyridin-4-ylmethyl{(1S)-1-methyl-2-[methyl(2-phenylethyl)amino]-2-oxoethyl}-carbamate;
pyridin-4-ylmethyl{(1S)-1-benzyl-2-[methyl(2-phenylethyl)amino]-2-oxoethyl}-carbamate;
pyridin-4-ylmethyl[(1S)-1-benzyl-2-(dimethylamino)-2-oxoethyl]carbamate;
pyridin-4-ylmethyl{(1S)-1-benzyl-2-[(3-methylbutyl)amino]-2-oxoethyl}carbamate;
pyridin-4-ylmethyl{(1S)-1-benzyl-2-[isopropyl(methyl)amino]-2-oxoethyl}carbamate;
pyridin-4-ylmethyl{(1S)-1-benzyl-2-[(3,3-dimethyl-2-oxobutyl)amino]-2-oxoethyl}-carbamate;
pyridin-4-ylmethyl{(1S)-1-benzyl-2-[(2,2-difluoroethyl)amino]-2-oxoethyl}carbamate;
pyridin-4-ylmethyl((1S)-1-benzyl-2-oxo-2-{[(2S)-tetrahydrofuran-2-ylmethyl]amino}-ethyl)carbamate;
pyridin-4-ylmethyl((1S)-1-benzyl-2-oxo-2-{[(2R)-tetrahydrofuran-2-ylmethyl]amino}-ethyl)carbamate;
pyridin-4-ylmethyl[(1S)-1-benzyl-2-morpholin-4-yl-2-oxoethyl]carbamate;
pyridin-4-ylmethyl{(1S)-1-(4-hydroxybenzyl)-1-methyl-2-[(3-methylbutyl)amino]-2-oxoethyl}carbamate;
pyridin-4-ylmethyl[(1S)-2-(benzylamino)-1-(4-hydroxybenzyl)-1-methyl-2-oxoethyl]-carbamate;
pyridin-4-ylmethyl((1S)-1-(4-hydroxybenzyl)-1-methyl-2-oxo-2-{[(1S)-1-phenyl-ethyl]amino}ethyl)carbamate;
pyridin-4-ylmethyl{(1S)-1-(4-hydroxybenzyl)-1-methyl-2-[methyl(2-phenylethyl)-amino]-2-oxoethyl}carbamate;
pyridin-4-ylmethyl{(1S)-1-benzyl-1-methyl-2-[(3-methylbutyl)amino]-2-oxoethyl}-carbamate;
pyridin-4-ylmethyl{1,1-dimethyl-2-[(3-methylbutyl)amino]-2-oxoethyl}carbamate;
(2,6-dimethylpyridin-4-yl)methyl{1,1-dimethyl-2-[(3-methylbutypamino]-2-oxo-ethyl}carbamate;
(2,6-dimethylpyridin-4-yl)methyl{1,1-dimethyl-2-[methyl(3-methylbutyl)amino]-2-oxoethyl}carbamate;
(2,6-dimethylpyridin-4-yl)methyl(1,1-dimethyl-2-morpholin-4-yl-2-oxoethyl)-carbamate;
(2,6-dimethylpyridin-4-yl)methyl{2-[(2R,65)-2,6-dimethylmorpholin-4-yl]-1,1-dimethyl-2-oxoethyl}carbamate;
(2,6-dimethylpyridin-4-yl)methyl{(1S)-1-(4-hydroxybenzyl)-1-methyl-2-[(3-methyl-butyl)amino]-2-oxoethyl}carbamate; and
(2,6-dimethylpyridin-4-yl)methyl[(1S)-1-(4-hydroxybenzyl)-1-methyl-2-morpholino-4-yl-2-oxoethyl]carbamate.

Another aspect of the present disclosure is a compound of formula (I) for use in therapy.

In a further aspect, the invention relates to a compound of formula (I) for use in the treatment or prevention of any of the disorders or conditions described herein.

In a yet further aspect, the invention relates to the use of the compounds of formula (I) in the manufacture of a medicament for the treatment or prevention of any of the disorders or conditions described herein.

In some embodiments, said compounds may be used for the treatment or prevention of a condition that is prevented, treated, or ameliorated by selective action via the leptin receptor.

In some embodiments, compounds of formula (I) may be used for the treatment or prevention of conditions (in particular, metabolic conditions) that are associated with weight gain. Conditions associated with weight gain include diseases, disorders, or other conditions that have an increased incidence in obese or overweight subjects. Examples include: lipodystrophy, HIV lipodystrophy, diabetes (type 2), insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain, macular degeneration. In some embodiments, compounds of the invention may also be used in the manufacture of a medicament for maintaining weight loss of a subject.

In some embodiments, compounds of formula (I) which are leptin receptor agonist mimetics may also be used to promote wound healing.

In some embodiments, compounds of formula (I) which are leptin receptor agonist mimetics may also be used for the treatment or prevention of conditions that cause a decrease in circulating leptin concentrations, and the consequent malfunction of the immune and reproductive systems. Examples of such conditions and malfunctions include severe weight loss, dysmenorrhea, amenorrhea, female infertility, immunodeficiency and conditions associated with low testosterone levels.

In some embodiments, compounds of formula (I) which are leptin receptor agonist mimetics may also be used for the treatment or prevention of conditions caused as a result of leptin deficiency, or a leptin or leptin receptor mutation.

In some other embodiments, compounds of formula (I) which are leptin receptor antagonist mimetics may be used for the treatment or prevention of inflammatory conditions or diseases, low level inflammation associated with obesity and excess plasma leptin and in reducing other complications associated with obesity including atherosclerosis, and for the correction of insulin resistance seen in Metabolic Syndrome and diabetes.

In some embodiments, compounds of formula (I) which are leptin receptor antagonist mimetics can be used for the treatment or prevention of inflammation caused by or associated with: cancer (such as leukemias, lymphomas, carcinomas, colon cancer, breast cancer, lung cancer, pancreatic cancer, hepatocellular carcinoma, kidney cancer, melanoma, hepatic, lung, breast, and prostate metastases, etc.); auto-immune disease (such as organ transplant rejection, lupus erythematosus, graft v. host rejection, allograft rejections, multiple sclerosis, rheumatoid arthritis, type I diabetes mellitus including the destruction of pancreatic islets leading to diabetes and the inflammatory consequences of diabetes); autoimmune damage (including multiple sclerosis, Guillam Bane Syndrome, myasthenia gravis); cardiovascular conditions associated with poor tissue perfusion and inflammation (such as atheromas, atherosclerosis, stroke, ischaemia-reperfusion injury, claudication, spinal cord injury, congestive heart failure, vasculitis, haemorrhagic shock, vasospasm following subarachnoid haemorrhage, vasospasm following cerebrovascular accident, pleuritis, pericarditis, the cardiovascular complications of diabetes); ischaemia-reperfusion injury, ischaemia and associated inflammation, restenosis following angioplasty and inflammatory aneurysms; epilepsy, neurodegeneration (including Alzheimer's Disease), arthritis (such as rheumatoid arthritis, osteoarthritis, rheumatoid spondylitis, gouty arthritis), fibrosis (for example of the lung, skin and liver), multiple sclerosis, sepsis, septic shock, encephalitis, infectious arthritis, Jarisch-Herxheimer reaction, shingles, toxic shock, cerebral malaria, Lyme's disease, endotoxic shock, gram negative shock, haemorrhagic shock, hepatitis (arising both from tissue damage or viral infection), deep vein thrombosis, gout; conditions associated with breathing difficulties (e.g. chronic obstructive pulmonary disease, impeded and obstructed airways, bronchoconstriction, pulmonary vasoconstriction, impeded respiration, chronic pulmonary inflammatory disease, silicosis, pulmonary sarcosis, cystic fibrosis, pulmonary hypertension, pulmonary vasoconstriction, emphysema, bronchial allergy and/or inflammation, asthma, hay fever, rhinitis, vernal conjunctivitis and adult respiratory distress syndrome); conditions associated with inflammation of the skin (including psoriasis, eczema, ulcers, contact dermatitis); conditions associated with inflammation of the bowel (including Crohn's disease, ulcerative colitis and pyresis, irritable bowel syndrome, inflammatory bowel disease); HIV (particularly HIV infection), cerebral malaria, bacterial meningitis, osteoporosis and other bone resorption diseases, osteoarthritis, infertility from endometriosis, fever and myalgia due to infection, and other conditions mediated by excessive anti-inflammatory cell (including neutrophil, eosinophil, macrophage and T-cell) activity.

In some embodiments, compounds of formula (I) which are leptin receptor antagonists mimetics may be used for the treatment or prevention of macro or micro vascular complications of type 1 or 2 diabetes, retinopathy, nephropathy, autonomic neuropathy, or blood vessel damage caused by ischaemia or atherosclerosis.

In some embodiments, compounds of formula (I) which are leptin receptor antagonist mimetics may be used to inhibit angiogenesis. Compounds that inhibit angiogenesis may be used for the treatment or prevention of obesity or complications associated with obesity. Compounds that inhibit angiogenesis may be used for the treatment or prevention of complications associated with inflammation diabetic retinopathy, or tumour growth particularly in breast, prostate or gastrointestinal cancer.

In a further aspect, the invention relates to a method for the treatment or prevention of any of the disorders or conditions described herein, which includes administering to a subject (e.g., a subject in need thereof, e.g., a mammal) an effective amount of a compound of formula I.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is prescreened or identified as in need of such treatment by assessment for a relevant marker or indicator of suitability for such treatment.

In one embodiment, the invention provides a method of monitoring treatment progress. The method includes the step of determining a level of diagnostic marker (Marker) (e.g., any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In certain method embodiments, a level of Marker or Marker activity in a subject is determined at least once. Comparison of Marker levels, e.g., to another measurement of Marker level obtained previously or subsequently from the same patient, another patient, or a normal subject, may be useful in determining whether therapy according to the disclosure is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, ELISA, radio labeling/assay techniques, blotting/chemiluminescence methods, real-time PCR, and the like.

In some embodiments, it may be advantageous if a compound of formula (I) is able to penetrate the central nervous system. In other embodiments, it may be advantageous if a is compound of formula (I) is not able to penetrate the CNS. In general, it is expected that compounds that are leptin receptor agonist mimetics may be particularly useful for the treatment or prevention of obesity, insulin resistance, or diabetes (particularly glucose intolerance) if these compounds can penetrate the CNS. A person of ordinary skill in the art can readily determine whether a compound can penetrate the CNS. A suitable method that may be used is described in the Biological Methods section.

A leptin receptor response may be measured in any suitable way. In vitro, this may be done be measuring leptin receptor signaling. For example, phosphorylation of Akt, STAT3, STAT5, MAPK, shp2 or the leptin receptor in response to binding of leptin or a compound of the invention to the leptin receptor may be measured. The extent of phosphorylation of Akt, STAT3, STAT5, MAPK, shp2 or the leptin receptor may be determined for example by Western blotting or by ELISA. Alternatively, a STAT reporter assay may be used, for example STAT driven luciferase expression. A cell line expressing the leptin receptor may be used for such assays. In vivo, leptin receptor response may be measured by determining the reduction in food intake and body weight after administration of leptin or a compound of formula (I).

The Biological Methods below describe assays and methods that can be used to determine whether a compound of formula (I) is a leptin receptor agonist mimetic or a leptin receptor antagonist mimetic.

A compound of formula (I) may be administered with or without other therapeutic agents. For example, where it is desired to reduce inflammation, a compound may be administered with an anti-inflammatory agent (for example, disease modifying anti-rheumatic drugs such as methotrexate, sulphasalazine and cytokine inactivating agents, steroids, NSAIDs, cannabinoids, tachykinin modulators, or bradykinin modulators). Where it is desired to provide an anti-tumour effect, a compound may be administered with a cytotoxic agent (for example, methotrexate, cyclophosphamide) or another anti-tumour drug.

Compounds of formula (I) may be radio labeled (for example with tritium or radioactive iodine) for in vitro or in vivo applications, such as receptor displacement studies or receptor imaging.

Definitions

The following definitions shall apply throughout the specification and the appended claims.

Unless otherwise stated or indicated, the term “C₁₋₆-alkyl” denotes a straight or branched alkyl group having from 1 to 6 carbon atoms. Examples of said C₁₋₆-alkyl include methyl, ethyl, n-propyl, iso-propyl, n-butyl, iso-butyl, sec-butyl, t-butyl, and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc.

Unless otherwise stated or indicated, the term “C₁₋₄-alkoxy” denotes a straight or branched alkoxy group having from 1 to 4 carbon atoms. Examples of said C₁₋₄-alkoxy include methoxy, ethoxy, n-propoxy, iso-propoxy, n-butoxy, iso-butoxy, sec-butoxy and t-butoxy. For parts of the range “C₁₋₄-alkoxy” all subgroups thereof are contemplated such as C₁₋₃-alkoxy, C₁₋₂-alkoxy, C₂₋₄-alkoxy, C₂₋₃-alkoxy and C₃₋₄-alkoxy.

Unless otherwise stated or indicated, the term “hydroxy-C₁₋₄-alkyl” denotes a straight or branched C₁₋₄-alkyl group that has a hydrogen atom thereof replaced with OH. Examples of said hydroxy-C₁₋₄-alkyl include hydroxymethyl and 2-hydroxyethyl.

Unless otherwise stated or indicated, the term “phenyl-C₁₋₆-alkyl” denotes a straight or branched C₁₋₆-alkyl group that has a hydrogen atom thereof replaced with phenyl. Examples of said phenyl-C₁₋₆-alkyl include phenylmethyl (i.e., benzyl), 1-phenylethyl and 2-phenylethyl.

Unless otherwise stated or indicated, the term “heterocyclyl-C₁₋₆-alkyl” denotes a straight or branched C₁₋₆-alkyl group that has a hydrogen atom thereof replaced with a fully saturated or partially unsaturated monocyclic ring having 3 to 8 ring atoms with at least one heteroatom such as O, N, or S, and the remaining ring atoms are carbon. Examples of said heterocyclyl-C₁₋₆-alkyl include tetrahydrofuran-2-ylmethyl, pyrrolidin-2-ylmethyl and piperazin-1-ylethyl.

When substituents R⁴ and R⁵ described herein, together with the nitrogen atom to which they are bound, form a saturated heterocyclic ring, said ring can be a 5- to 7-membered ring and optionally contain one or more additional heteroatoms selected from O, S and N. Examples of such heterocyclic rings include piperidine, piperazine and morpholine.

The term “oxo” denotes

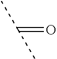

“Halogen” refers to fluorine, chlorine, bromine or iodine.

“Hydroxy” refers to the —OH radical.

“Cyano” refers to the —CN radical.

“Optional” or “optionally” means that the subsequently described event or circumstance may but need not occur, and that the description includes instances where the event or circumstance occurs and instances in which it does not.

The term “mammal” includes organisms, which include mice, rats, cows, sheep, pigs, rabbits, goats, and horses, monkeys, dogs, cats, and preferably humans. The subject may be a human subject or a non human animal, particularly a domesticated animal, such as a dog.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect).

“Prodrugs” refers to compounds that may be converted under physiological conditions or by solvolysis to a biologically active compound of formula (I). A prodrug may be inactive when administered to a subject in need thereof, but is converted in vivo to an active compound of formula (I). Prodrugs are typically rapidly transformed in vivo to yield the parent compound, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2^{nd} Ed., Elsevier Academic Press (2004), pp. 498-549). Prodrugs may be prepared by modifying functional groups, such as a hydroxy, amino or mercapto groups, present in a compound of formula (I) in such a way that the modifications are cleaved, either in routine manipulation or in vivo, to the parent compound. Examples of prodrugs include, but are not limited to, acetate, formate and succinate derivatives of hydroxy functional groups or phenyl carbamate derivatives of amino functional groups.

Throughout the specification and the appended claims, a given chemical formula or name shall also encompass all salts, hydrates, solvates, N-oxides and prodrug forms thereof. Further, a given chemical formula or name shall encompass all tautomeric and stereoisomeric forms thereof. Stereoisomers include enantiomers and diastereomers. Enantiomers can be present in their pure forms, or as racemic (equal) or unequal mixtures of two enantiomers. Diastereomers can be present in their pure forms, or as mixtures of diastereomers. Diastereomers also include geometrical isomers, which can be present in their pure cis or trans forms or as mixtures of those.

The compounds of formula (I) may be used as such or, where appropriate, as pharmacologically acceptable salts (acid or base addition salts) thereof The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

Compositions

For clinical use, the compounds of formula (I) are formulated into pharmaceutical formulations for various modes of administration. It will be appreciated that the compounds may be administered together with a physiologically acceptable carrier, excipient, or diluent. The pharmaceutical compositions may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration.

Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glyco late, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, the compounds may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

Preparation of Compounds of the Invention

The compounds of formula (I) above may be prepared by, or in analogy with, conventional methods. Formation of the urethane and the amide linkers are the key synthetic steps in preparing the compounds of formula (I). A large number of activating agents can be used for the formation of a urethane linker, e.g. phosgene to form the chloroformate of an alcohol, or carbonyldiimidazole (CDI) to form an imidazole carboxylate. Typically the urethane linkers incorporated into compounds of formula (I) have been synthesized utilizing triphosgene or bis-(4-nitrophenyl)carbonate as the activating agent. Activating agents that can be used for the formation of an amide linker include thionyl chloride, N,N′-disuccinimidyl carbonate (DSC), N,N′-dicyclohexylcarbodiimide (DCC), PyBrOP, HBTU, TBTU and HCTU. Typically the amide linkers incorporated into compounds of formula (I) have been synthesized utilizing PyBrOP, HBTU or HCTU as the activating agent. The preparation of intermediates and compounds according to the examples of the present invention may in particular be illuminated by the following Schemes 1-3. Definitions of variables in the structures in the schemes herein are commensurate with those of corresponding positions in the formulae delineated herein.

Compounds of formula (I′) wherein A is piperazinyl and Y is O can easily be prepared in only a few steps as shown in Scheme 1 below. In the first step, a suitably protected alcohol derivative of formula (II) is activated with triphosgene in the presence of a base (such as DMAP) in an aprotic solvent (such as DCM) to give the corresponding chloroformate of formula (III). The chloroformate intermediate (III) is then subsequently treated with the appropriate amine of formula (IV) in the presence of a base (such as DMAP) in an aprotic solvent (such as DCM), resulting in the formation of the desired urethane linker, to give the compound of formula (V). The formation of the urethane is typically a two step process but this may also be performed in a one-pot reaction by formation of the activated intermediate in situ. Removal of the protecting group R⁸ gives the corresponding carboxylic acid of formula (VI). Treatment of (VI) with an activating reagent (such as PyBrOP or HBTU) and subsequent addition of the appropriate amine of formula (VII) in the presence of a base (such as DIPEA) in an aprotic solvent (such as DMF) affords the amide linker present in a compound of formula (VIII). In the final step, the protecting group R⁷ is removed, resulting in the formation of the desired compound of formula (I′).

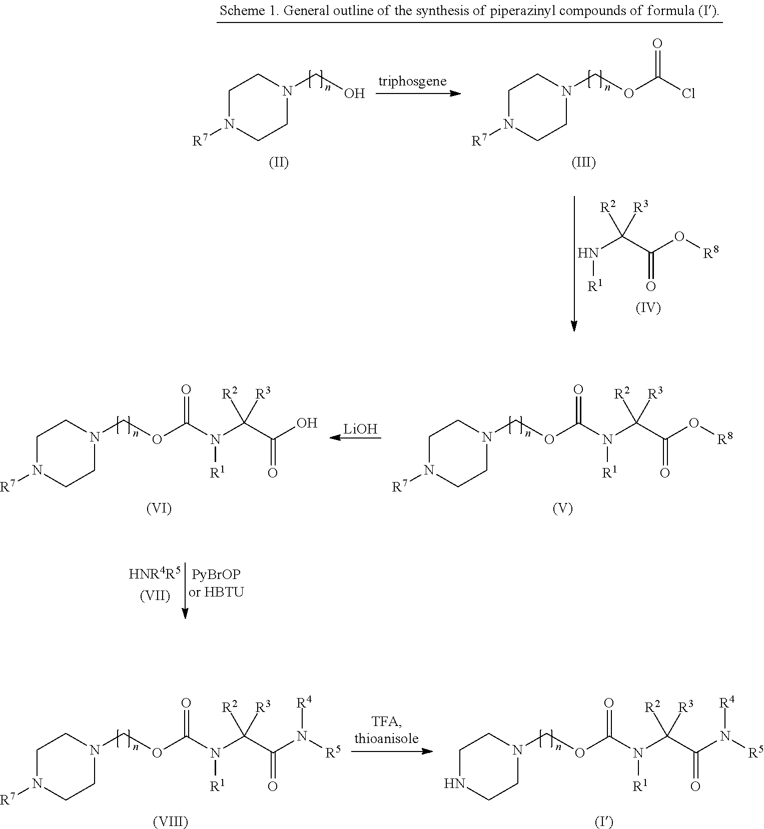

wherein R¹, R², R³, R⁴, R⁵ and n are as defined in formula (I);

R⁷ is an N-protecting group (e.g. Boc); and

R⁸ is a protecting group (e.g. methyl).

Scheme 2 shows a related procedure for the preparation of compounds of formula (I″) wherein A is pyridinyl and Y is O. In the first step, an alcohol derivative of formula (IX) is treated with bis-(4-nitrophenyl)carbonate in the presence of a base (such as NMM) in an aprotic solvent (such as DCM) to give the corresponding carbonate of formula (X). Formation of the urethane linker is achieved by treatment of the carbonate intermediate (X) with the appropriate amine of formula (IV) in the presence of a base (such as DIPEA) and an activating agent (such as DMAP) in an aprotic solvent (such as DMF), resulting in a compound of formula (XI). The formation of the urethane is typically a two step process but this may also be performed in a one-pot reaction by formation of the activated intermediate in situ. Removal of the protecting group R⁸ then gives the corresponding carboxylic acid of formula (XII). Treatment of the carboxylic acid (XII) with an activating reagent (such as PyBrOP, TBTU, HCTU or HBTU) and subsequent addition of the appropriate amine of formula (VII) and a base (such as DIPEA) in an aprotic solvent (such as DMF) finally results in the formation of the desired compound of formula (I″).

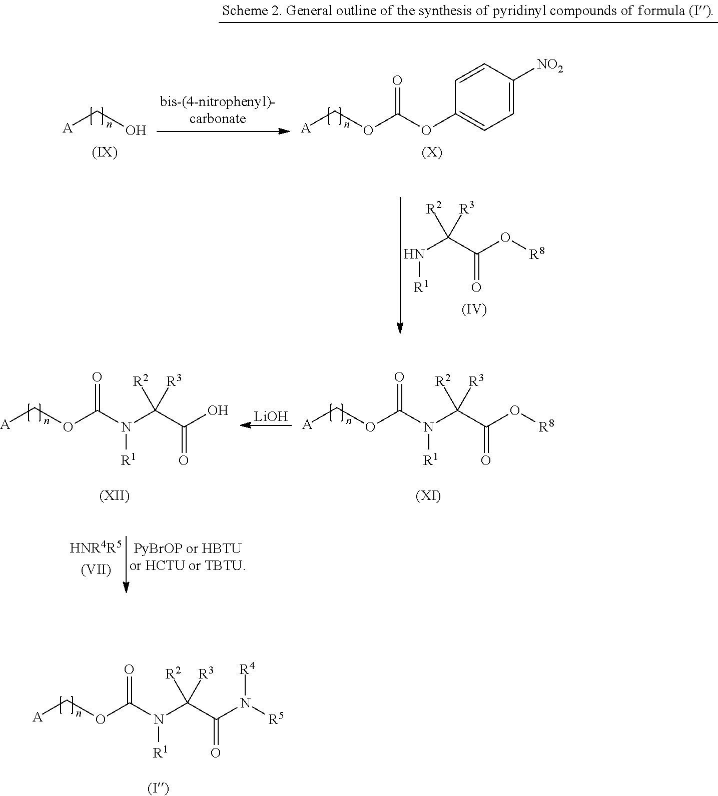

wherein A is pyridinyl;

R¹, R², R³, R⁴, R⁵ and n are as defined in formula (I); and

R⁸ is a protecting group.

Alternatively, compounds of formula (I″) wherein A is pyridinyl and Y is O can easily be prepared by forming the amide linker first and then the urethane linker as shown in Scheme 3 below. In the first step, a suitably N-protected compound of formula (XIII) is treated with an activating reagent (such as PyBrOP or TBTU) followed by the addition of the appropriate amine of formula (VII) in the presence of a base (such as DIPEA) in an aprotic solvent (such as DMF), resulting in formation of the amide intermediate of formula (XIV). Removal of the protecting group R⁹ then gives the corresponding amine intermediate of formula (XV). Subsequent treatment of (XV) with carbonate intermediate (X) in the presence of a base (such as DIPEA) and an activating agent (such as DMAP) in an aprotic solvent (such as DMF) results in formation of the urethane linker to give a compound of formula (I″).

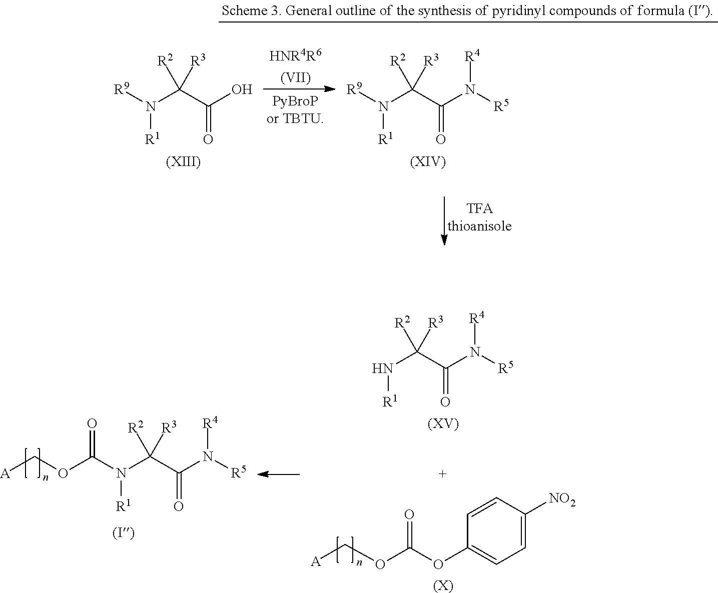

wherein A is pyridinyl;

R¹, R², R³, R⁴, R⁵ and n are as defined in formula (I); and

R⁹ is an N— protecting group (e.g. Boc).

The necessary starting materials for preparing the compounds of formula (I) are either commercially available, or may be prepared by methods known in the art.

The processes described below in the experimental section may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g., as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. Examples of protecting groups are t-butoxycarbonyl (Boc), benzyl and trityl (triphenylmethyl). The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The following abbreviations have been used:

| | |
|---|---|
| aq | Aqueous |
| Boc | tert-Butoxy carbonyl |
| DCM | Dichloromethane |
| DIPEA | N,N-Diisopropylethylamine |
| DMAP | N,N-Dimethylaminopyridine |
| DMF | N,N-Dimethylformamide |
| ES⁺ | Electrospray |
| Et₂O | Diethyl ether |
| EtOAc | Ethyl acetate |
| HIV | Human immunodeficiency virus |
| HBTU | 2-(1H-Benzotriazole-1-yl)-1,1,3,3-tetramethylaminium |
| | hexafluorophosphate |
| HCTU | 2-(6-Chloro-1H-Benzotriazole-1-yl)-1,1,3,3-tetramethylaminium |
| | hexafluorophosphate |
| HPLC | High performance liquid chromatography |
| ICV | Intracerebroventricular |
| LCMS | Liquid Chromatography Mass Spectrometry |
| M | Molar |
| [MH]⁺ | Protonated molecular ion |
| NEt₃ | Triethylamine |
| NMM | N-methyl morpholine |
| PyBrOP | Bromo-tris-pyrrolidino-phosphonium hexafluorophosphate |
| RP | Reverse Phase |
| sat | Saturated |
| r.t. | Room temperature |
| tert | Tertiary |
| TFA | Trifluoroacetic acid |
| THF | Tetrahydrofuran |
| TBTU | 2-(1H-Benzotriazole-1-yl)-1,1,3,3-tetramethylaminium |
| | tetrafluoroborate |

Embodiments of the disclosure are described in the following examples with reference to the accompanying drawings, in which:

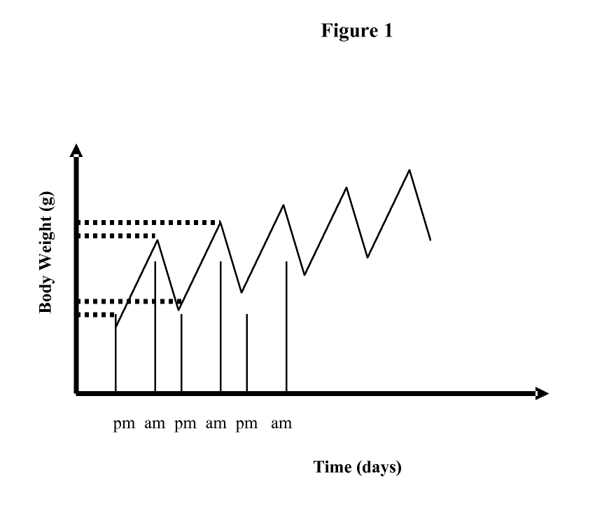

FIG. 1 is a schematic drawing illustrating weight gain and weight loss in mice during dark and light phases, respectively. The graph illustrates the large nocturnal weight increase versus the comparatively small body weight change over 24 hours

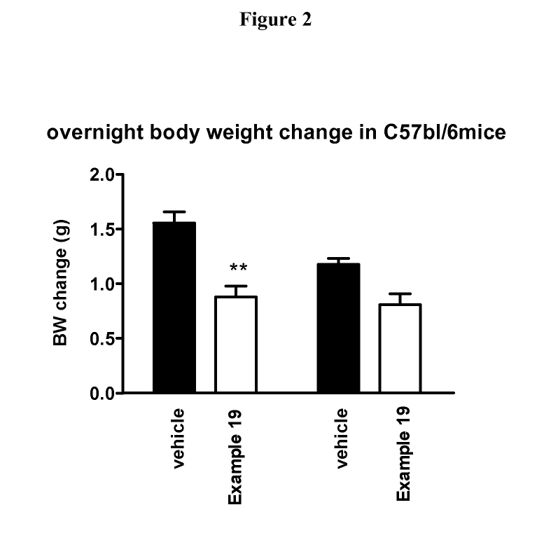

FIG. 2 shows the effect of Example 19 on the body weight in mice between the beginning of the dark phase and the beginning of the light phase (pm-am).

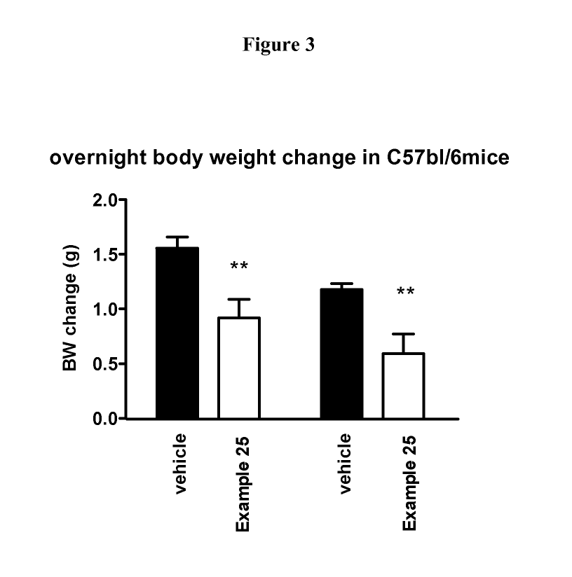

FIG. 3 shows the effect of Example 25 on the body weight in mice between the beginning of the dark phase and the beginning of the light phase (pm-am).

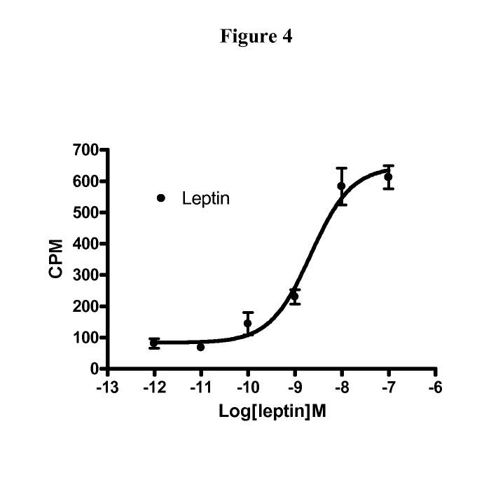

FIG. 4 shows the concentration-dependent increase in [³H]-thymidine incorporation by JEG-3 cells for leptin

The recitation of a listing of chemical groups in any definition of a variable herein includes definitions of that variable as any single group or combination of listed groups. The recitation of an embodiment herein includes that embodiment as any single embodiment or in combination with any other embodiments or portions thereof.

is The disclosure will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All references and publications cited herein are hereby incorporated by reference in their entirety.

### EXAMPLES AND INTERMEDIATE COMPOUNDS

Experimental Methods

All reagents were commercial grade and were used as received without further purification, unless otherwise specified. Commercially available anhydrous solvents were used for reactions conducted under inert atmosphere. Reagent grade solvents were used in all other cases, unless otherwise specified. Analytical LCMS was performed on a Waters ZQ mass spectrometer connected to an Agilent 1100 HPLC system. Analytical HPLC was performed on an Agilent 1100 system or Schimadzu CLASS-VP system. High-resolution mass spectra (HRMS) were obtained on an Agilent MSD-TOF connected to an Agilent 1100 HPLC system. During the analyses the calibration was checked by two masses and automatically corrected when needed. Spectra are acquired in positive electrospray mode. The acquired mass range was m/z 100-1100. Profile detection of the mass peaks was used. Normal phase chromatography was performed on a Flash Master Personal system equipped with 20 g Strata SI-1 silica gigatubes. Reverse phase chromatography was performed on a Gilson system equipped with Merck LiChoprep® RP-18 (40-63 μm) 460×26 mm column, 30 mL/min, gradient of methanol in water from 0% to 100%. Preparative HPLC was performed on a Gilson system equipped with Phenomenex Hydro RP 150×20 mm, 20 mL/min, gradient of acetonitrile in water from 0% to 100%. The compounds were automatically named using ACD 6.0.

Analytical HPLC and LCMS data were obtained with:

System A: Phenomenex Synergi Hydro RP (50×4.6 mm, 4 μm), gradient 5-100% CH₃CN in H₂O (+0.1% HCO2H), 1.0 mL/min, gradient time 3 min, 200-300 nm, 25° C.;

System B: Phenomenex Synergi Hydro RP (150×4.6 mm, 4 μ1m), gradient 5-100% CH₃CN in H₂O (+0.1% HCO₂H), 1.0 mL/min, gradient time 8 min, 25° C.;

System C: Phenomenex Synergi Hydro RP (150×4.6 mm, 4 μm), gradient 5-100% CH₃CN (+0.085% TFA) in H₂O (+0.1% TFA), 1.0 mL/min, gradient time 7 min, 25° C.;

System D: Phenomenex Synergi Hydro RP (150×4.6 mm, 4 μm), gradient 5-100% CH₃CN (+0.085% TFA) in H₂O (+0.1% TFA), 1.5 mL/min, gradient time 10 min, 200-300 nm, 25° C.;

System E: Phenomenex Synergi Hydro RP (150×4.6 mm, 4 μm), gradient 5-100% CH₃CN (+0.085% TFA) in H₂O (+0.1% TFA), 1.5 mL/min, gradient time 7 min, 200-300 nm, 30° C.;

4-Nitrophenyl (pyridin-4-yl)methyl carbonate was prepared according to the procedure described by Veber, D. F., J. Org. Chem., 1977, 42, 3280. The last two steps in Example 32, converting (S)-2-(4-hydroxy-3,5-diiodobenzyl)-2-amino-N-isopentylpropanamide to the final tritiated compound (pyridin-4-yl)methyl(S)-2-(isopentylcarbamoyl)-1-(3,5-ditritium-4-hydroxyphenyl)propan-2-ylcarbamate, were performed by the Tritium Custom Preparations Group, Amersham Biosciences, The Maynard Centre, Forest Farm Estate, Whitchurch, Cardiff, CF14 7YT.

Intermediate 1

### 2-(4-(tert-Butoxycarbonyl)piperazin-1-yl)ethyl(S)-1-(carboxy)-2-(4-tert-butoxy-phenyl)ethylcarbamate

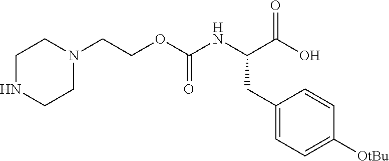

### Step 1: tert-Butyl 4-(2-hydroxyethyl)piperazine-1-carboxylate

To a solution of 1-(2-hydroxyethyl)piperazine (51.7 g, 398 mmol) in DCM (500 mL) was added NEt₃ (70.0 mL, 526 mmol) and di-tert-butyl dicarbonate (80.0 g, 367 mmol). The reaction mixture was stirred overnight at r.t. and then washed with 1M aq Na₂CO₃ solution (2×300 mL). The organic phase was dried (MgSO₄) and concentrated in vacuo to give tert-butyl 4-(2-hydroxyethyl)piperazine-1-carboxylate (66.0 g, 72%) as a colourless oil.

### Step 2: 2-(4-(tert-Butoxycarbonyl)piperazin-1-yl)ethyl(S)-1-(methoxycarbonyl)-2-(4-tert-butoxyphenyl)ethylcarbamate

Triphosgene (618 mg, 2.08 mmol) was dissolved in DCM (30 mL) and a solution of tert-butyl 4-(2-hydroxyethyl)piperazine-1-carboxylate (1.43 g, 6.21 mmol) and DMAP (750 mg, 6.15 mmol) in DCM (10 mL) was added. The reaction mixture was stirred at r.t. for 4 hours. A solution of (S)-methyl 2-amino-3-(4-tert-butoxyphenyl)propanoate hydrochloride (1.79 g, 6.22 mmol) and DMAP (1.50 g, 12.3 mmol) in DCM (10 mL) was added. The reaction mixture was stirred overnight, then poured onto a sat aq NaHCO₃ solution (100 mL) and extracted with DCM (3×100 mL). The combined organic layers were dried (MgSO₄) and concentrated in vacuo. The residue was purified by normal phase chromatography (gradient eluting with MeOH in DCM from 0% to 5%) to give 2-(4-(tert-butoxycarbonyl)piperazin-1-yl)ethyl(S)-1-(methoxycarbonyl)-2-(4-tert-butoxyphenyl)-ethylcarbamate (2.38 g, 76%) as a colourless oil.

### Step 3: 2-(4-(tert-butoxycarbonyl)piperazin-1-yl)ethyl(S)-1-(carboxy)-2-(4-tert-butoxy-phenyl)ethylcarbamate

2-(4-(tert-Butoxycarbonyl)piperazin-1-yl)ethyl(S)-1-(methoxycarbonyl)-2-(4-tert-butoxy-phenyl)ethylcarbamate from the previous step (2.38 g, 4.7 mmol) was dissolved in THF (50 mL) and treated with a solution of LiOH.H₂O (580 mg, 13.8 mmol) in water (15 mL). The reaction mixture was stirred vigorously for six hours and then left to stand overnight. The reaction mixture was poured onto water (100 mL) and extracted with EtOAc (100 mL). The aqueous layer was acidified to pH 4 with dilute aq HCl solution, saturated with sodium chloride and extracted with EtOAc (3×100 mL). The combined organic layers were dried (MgSO₄) and concentrated in vacuo to give 2-(4-(tert-butoxycarbonyl)-piperazin-1-yl)ethyl(S)-1-(carboxy)-2-(4-tert-butoxyphenyl)ethylcarbamate (1.98 g, 85%).

Intermediate 2

### (Pyridin-4-yl)methyl(S)-1-(carboxy)-2-(4-hydroxyphenyl)ethylcarbamate

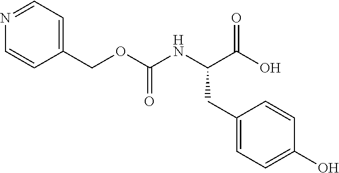

### Step 1: (Pyridin-4-yl)methyl(S)-1-(methoxycarbonyl)-2-(4-hydroxyphenyl)ethylcarbamate

4-Nitrophenyl (pyridin-4-yl)methyl carbonate (835 mg, 3.0 mmol), (S)-tyrosine methyl ester (508 mg, 2.6 mmol), DIPEA (0.50 mL, 2.9 mmol) and DMAP (20 mg) were dissolved in DMF (20 mL) and stirred for 18 hours. The reaction mixture was concentrated in vacuo. The residue was suspended in sat aq NaHCO₃ solution (50 mL) and extracted with EtOAc (2×50 mL). The combined organic layers were washed with sat aq NaHCO₃ solution (5×50 mL), dried (MgSO₄) and concentrated in vacuo. The residue was purified by normal phase chromatography (gradient eluting with MeOH in DCM from 0% to 5%) to give (pyridin-4-yl)methyl(S)-1-(methoxycarbonyl)-2-(4-hydroxyphenyl)ethylcarbamate (817 mg, 96%) as a white foam.

### Step 2: (Pyridin-4-yl)methyl(S)-1-(carboxy)-2-(4-hydroxyphenyl)ethylcarbamate

(Pyridin-4-yl)methyl(S)-1-(methoxycarbonyl)-2-(4-hydroxyphenyl)ethylcarbamate (817 mg, 2.47 mmol) was dissolved in THF (30 mL) and treated with a solution of LiOH.H₂O (300 mg, 7.1 mmol) in water (6 mL) and stirred vigorously overnight. The reaction mixture was poured into water (50 mL) and the layers separated. The aqueous layer was acidified with 0.2M HCl and AcOH to pH ˜4 and then extracted with EtOAc (3×50 mL). The combined organic layers were dried (MgSO₄) and concentrated in vacuo to give (pyridin-4-yl)methyl (S)-1-(carboxy)-2-(4-hydroxyphenyl)ethylcarbamate compound (615 mg, 78%) as a white solid.

Intermediate 3

### (Pyridin-4-yl)methyl(S)-1-(carboxy)-2-phenylethylcarbamate

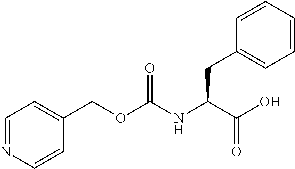

Phenylalanine methyl ester hydrochloride (4.00 g, 18.5 mmol), 4-nitrophenyl(pyridin-4-yl)methyl carbonate (4.62 g, 16.9 mmol), DIPEA (5.87 mL, 33.7 mmol) and DMAP (catalytic amount) were dissolved in DMF (70 mL). The reaction mixture was stirred at r.t. for 26 hours and then concentrated in vacuo. The residue was dissolved in EtOAc (100 mL) and washed with a 1M aq Na₂CO₃ solution. The EtOAc phase was concentrated in vacuo and the residue purified by normal phase chromatography (gradient eluting with MeOH in DCM from 0% to 5%) to give (pyridin-4-yl)methyl(S)-1-(methoxycarbonyl)-2-phenylethylcarbamate (4.29 g, 81%) as a yellow oil. The entirety of this material (4.29 g, 13.6 mmol) was dissolved in THF (90 mL) and a solution of LiOH.H₂O (1.72 g, 41.0 mmol) in water (30 mL) was added. The reaction was stirred at r.t. for 4 hours and then quenched with 1M HCl (41.0 mL, 41.0 mmol). The THF was removed in vacuo and a white solid crystallised from the aqueous layer. The solid was collected by filtration and dried in vacuo to give (pyridin-4-yl)methyl(S)-1-(carboxy)-2-phenylethylcarbamate (3.40 g, 83%) as a white crystalline solid.

Intermediate 4

### (Pyridin-4-yl)methyl(S)-2-(carboxy)-1-(4-hydroxyphenyl)propan-2-ylcarbamate

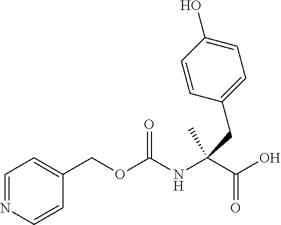

To a solution of (S)-methyl 2-(4-hydroxybenzyl)-2-aminopropanoate hydrochloride (0.59 g, 2.4 mmol) in DMF (15 mL) was added DIPEA (1.26 ml, 7.2 mmol), DMAP (30 mg) and then 4-nitrophenyl (pyridin-4-yl)methyl carbonate (0.65 g, 2.4 mmol). The reaction was stirred overnight at r.t. The DMF was removed in vacuo and the residue dissolved in EtOAc (40 mL), washed with 1M aq Na₂CO₃ (6×40 mL), dried (Na₂SO₄), filtered and evaporated to dryness. The residue was purified by normal phase chromatography (gradient eluting with MeOH in DCM from 0% to 4%) to give (pyridin-4-yl)methyl(S)-2-(methoxycarbonyl)-1-(4-hydroxyphenyl)propan-2-ylcarbamate (379 mg, 46%) as a transparent oil that solidified on standing. The entirety of this material (379 mg, 1.1 mmol) was dissolved in THF (13 mL) and a 1M aq solution of LiOH (3.3 mL, 3.3 mmol) was added. The reaction mixture was stirred overnight. After evaporation of the volatiles, the residue was dissolved in water (30 mL) and washed with DCM (3×20 mL). The basic aqueous layer was then acidified to pH 4 with 5M HCl and extracted with EtOAc (6×30 mL). The combined EtOAc extracts were dried (Na₂SO₄), filtered and concentrated in vacuo to give (pyridin-4-yl)methyl(S)-2-(carboxy)-1-(4-hydroxyphenyl)propan-2-yl-carbamate (0.24 g, 66%) as a white solid.

Intermediate 5

### (2,6-Dimethylpyridin-4-yl)methyl 2-(carboxy)propan-2-ylcarbamate

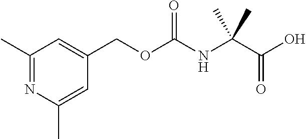

### Step 1: (2,6-Dimethylpyridin-4-yl)methyl 4-nitrophenyl carbonate

A suspension of (2,6-dimethyl-pyridin-4-yl)-methanol (9.14 g, 66.7 mmol) in DCM (40 mL) was added to a solution of bis-4-nitrophenylcarbonate (20.28 g, 66.7 mmol) in DCM (200 mL), followed by NMM (7.34 mL). The reaction mixture was stirred overnight and then washed with sat aq NaHCO₃ solution (5×100 mL). The DCM phase was dried (MgSO₄) and concentrated in vacuo. The residue was recrystallised from EtOAc (˜25 mL) to give (2,6-dimethylpyridin-4-yl)methyl 4-nitrophenyl carbonate (11.5 g, 57%) as an off-white solid. The filtrate from the crystallisation was concentrated in vacuo and the residue recrystallised from EtOAc (15 mL) with a drop of heptane to give an additional portion of (2,6-dimethylpyridin-4-yl)methyl 4-nitrophenyl carbonate (4.76 g, 24% yield-81% combined yield) as an off-white solid.

### Step 2: (2,6-Dimethylpyridin-4-yl)methyl 2-(methoxycarbonyl)propan-2-ylcarbamate

To a stirred solution of (2,6-dimethylpyridin-4-yl)methyl 4-nitrophenyl carbonate (5.62 g, 18.6 mmol), DIPEA (9.72 mL, 55.8 mmol) and DMAP (10 mg) in DMF (50 mL) was added aminoisobutyric acid methyl ester hydrochloride (3.00 g, 19.5 mmol). The reaction mixture was stirred at r.t. for 20 h and then evaporated in vacuo. The residue was dissolved in EtOAc (80 mL) and washed with multiple aliquots of 1M aq Na₂CO₃ solution until the aqueous layer was colourless. The organic layer was dried (MgSO₄), filtered and evaporated to dryness to give a white solid. Recrystallisation from EtOAc gave (2,6-dimethylpyridin-4-yl)methyl 2-(methoxycarbonyl)propan-2-ylcarbamate (2.58 g, 49%) as a white solid.

### Step 3: (2,6-Dimethylpyridin-4-yl)methyl 2-(carboxy)propan-2-ylcarbamate

(2,6-Dimethylpyridin-4-yl)methyl 2-(methoxycarbonyl)propan-2-ylcarbamate (2.58 g, 9.2 mmol) was dissolved in THF (60 mL) and a 1M aq solution of LiOH (27.6 mL, 27.6 mmol) was added. The reaction was stirred for 3 hours before quenching with 1M aq HCl (27.6 mL, 27.6 mmol). After evaporation of the volatiles, the residue was added to a mixture of DCM (98 mL) and MeOH (2 mL) and filtered. The filtrate was dried in vacuo to give (2,6-dimethylpyridin-4-yl)methyl 2-(carboxy)propan-2-ylcarbamate (1.50 g, 61%) as a white solid.

Intermediate 6

### (2,6-Dimethylpyridin-4-yl)methyl(S)-2-(carboxy)-1-(4-hydroxyphenyl)propan-2-yl-carbamate

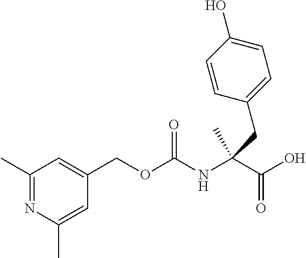

### Step 1: Methyl N,O-bis{[(2,6-dimethylpyridin-4-yl)methoxy]carbonyl}-α-methyl-L-tyrosinate

To a stirred solution of (2,6-dimethylpyridin-4-yl)methyl 4-nitrophenyl carbonate (11.5 g, 38.0 mmol), DIPEA (13.0 mL, 74.6 mmol) and DMAP (10 mg) in DMF (80 mL) was added alpha-methyl-tyrosine methyl ester hydrochloride (4.91 g, 20.0 mmol). The reaction to was stirred at r.t. for 20 h and then concentrated in vacuo. The residue was dissolved in EtOAc (80 mL), washed with multiple aliquots of 1M aq Na₂CO₃ solution until the aqueous layer was colourless, dried (MgSO₄), filtered and evaporated to dryness. The residue was recrystallised from EtOAc to give methyl N,O-bis{[(2,6-dimethylpyridin-4-yl)methoxy]carbonyl}-α-methyl-L-tyrosinate (1.80 g, 17%) as a pale yellow solid.

### Step 2: (2,6-dimethylpyridin-4-yl)methyl(S)-2-(carboxy)-1-(4-hydroxyphenyl)propan-2-yl-carbamate

To a solution of methyl N,O-bis{[(2,6-dimethylpyridin-4-yl)methoxy]carbonyl}-α-methyl-L-tyrosinate (1.80 g, 3.4 mmol) in THF (40 mL) was added 1M aq LiOH solution (17.0 mL, 17.0 mmol). The reaction was stirred overnight, quenched with 1M aq HCl (17.0 mL, 17.0 mmol) and dried in vacuo to give (2,6-dimethylpyridin-4-yl)methyl(S)-2-(carboxy)-1-(4-hydroxyphenyl)propan-2-ylcarbamate (1.07 g, 89%).

Example 1

### 2-Piperazin-1-ylethyl{(1S)-1-(4-hydroxybenzyl)-2-[methyl(3-methylbutyl)amino]-2-oxoethyl}carbamate dihydrochloride

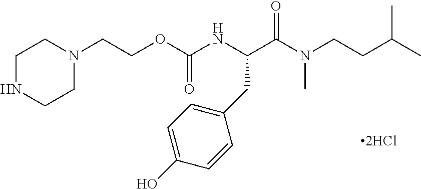

2-(4-(tert-Butoxycarbonyl)piperazin-1-yl)ethyl(S)-1-(carboxy)-2-(4-tert-butoxyphenyl)-ethylcarbamate (Intermediate 1; 415 mg, 0.84 mmol), N-methylisoamylamine (85 mg, 0.84 mmol) and DIPEA (0.40 mL, 2.30 mmol) were dissolved in DMF (10 mL) and then cooled in an ice-water bath. PyBrOP (400 mg, 0.86 mmol) was added. The reaction mixture was stirred at 0° C. for 6 hours and then allowed to warm to r.t. overnight. The reaction mixture was concentrated in vacuo. The residue was suspended in 0.2M aq HCl (50 mL) and extracted with DCM (3×50 mL). The combined DCM extracts were dried (MgSO₄), concentrated in vacuo and purified by reverse phase chromatography to give 2-(4-(tert-butoxycarbonyl)piperazin-1-yl)ethyl(S)-1-(N-isopentyl-N-methylcarbamoyl)-2-(4-tert-butoxyhydroxyphenyl)ethylcarbamate (262 mg, 54%) as a yellow gum. The entirety of this material (262 mg, 0.455 mmol) was dissolved in DCM (10 mL) and treated with thioanisole (0.4 mL) followed by TFA (3.0 mL). The reaction mixture was stirred overnight and then concentrated in vacuo. The residue was dissolved in 0.2M HCl in acetic acid (10 mL) and concentrated in vacuo. This procedure was repeated to ensure all the TFA was removed. The residue was triturated with Et₂O to give a white solid, which was purified by preparative HPLC (gradient eluting with acetonitrile in water from 5% to 100%) to give the title compound (115 mg, 51%) as a white foam.

Analytical HPLC: purity 98.4% (System D, R_{T}=5.96 min); Analytical LCMS: purity 100% (System A, R_{T}=2.40 min), ES⁺: 422.0 [MH]⁺; HRMS calcd for C₂₂H₃₆N₄O₄: 420.2737, found 420.2744.

### Example 2

### 2-Piperazin-1-ylethyl[(1S)-2-[benzyl(methyl)amino]-1-(4-hydroxybenzyl)-2-oxo-ethyl]carbamate dihydrochloride

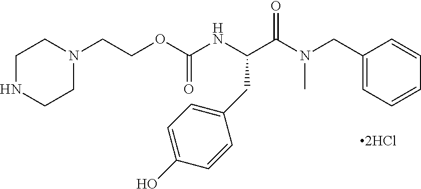

2-(4-(tert-Butoxycarbonyl)piperazin-1-yl)ethyl(S)-1-(carboxy)-2-(4-tert-butoxyphenyl)-ethylcarbamate (Intermediate 1; 378 mg, 0.77 mmol), N-methylbenzylamine (95 mg, 0.75 mmol), PyBrOP (360 mg, 0.77 mmol) and DIPEA (0.40 mL, 2.30 mmol) were dissolved in DMF (10 mL) cooled with an ice-water. The reaction mixture was stirred overnight and then concentrated in vacuo. The residue was suspended in 6% aq NaHCO₃ solution (50 mL) and extracted with DCM (3×50 mL). The combined DCM extracts were dried (MgSO₄) and concentrated in vacuo. The residue was purified by normal phase chromatography (gradient eluting with MeOH in DCM from 0% to 10%) followed by reverse phase chromatography to give 2-(4-(tert-butoxycarbonyl)piperazin-1-yl)ethyl(S)-1-(N-benzyl-N-methylcarbamoyl)-2-(4-tert-butoxyphenyl)ethylcarbamate (164 mg, 35%) as a yellow gum. This entirety of this material (164 mg, 0.27 mmol) was dissolved in DCM (10 mL) and treated with thioanisole (0.5 mL) followed by TFA (3 mL). The reaction mixture was stirred overnight and then concentrated in vacuo. The residue was dissolved in 0.2M HCl in acetic acid (10 mL) and concentrated in vacuo. This procedure was repeated to ensure all TFA was removed. The residue was triturated with Et₂O to give a white solid, which was purified by reverse phase chromatography to give the title compound (82 mg, 59%) as a white solid.

Analytical HPLC: purity 99.0% (System D, R_{T}=5.75 min); Analytical LCMS: purity 100% (System B, R_{T}=3.77 min), ES⁺: 441.8 [MH]⁺; HRMS calcd for C₂₄H₃₂N₄O₄: 440.2424, found 440.2435.

### Example 3

### 2-Piperazin-1-ylethyl{(1S)-1-(4-hydroxybenzyl)-2-[methyl(2-phenylethyl)amino]-2-oxoethyl}carbamate dihydrochloride

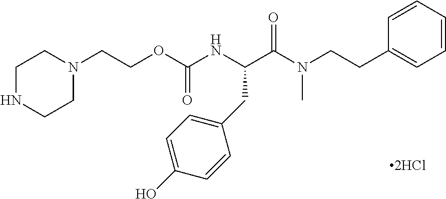

2-(4-(tert-Butoxycarbonyl)piperazin-1-yl)ethyl(S)-1-(carboxy)-2-(4-tert-butoxyphenyl)-ethylcarbamate (Intermediate 1; 404 mg, 0.82 mmol), N-methylphenethylamine (120 mg, 0.89 mmol), PyBrOP (390 mg, 0.84 mmol) and DIPEA (0.4 mL, 2.3 mmol) were dissolved in DMF (10 mL) cooled with an ice-water bath. The reaction mixture was stirred overnight and then concentrated in vacuo. The residue was suspended in 6% aq NaHCO₃ solution (50 mL) and extracted with DCM (3×50 mL). The combined DCM extracts were dried (MgSO₄) and concentrated in vacuo. The residue was purified by reverse phase chromatography to give 2-(4-(tert-butoxycarbonyl)piperazin-1-yl)ethyl(S)-1-(N-methyl-N-phenethylcarbamoyl)-2-(4-tert-butoxyphenyl)ethylcarbamate (257 mg, 51%) as a yellow gum. The entirety of this material (257 mg, 0.42 mmol) was dissolved in DCM (10 mL), treated with thioanisole (0.5 mL) followed by TFA (3 mL), stirred overnight and concentrated in vacuo. The residue was dissolved in 0.2M HCl in acetic acid (10 mL) and concentrated in vacuo. This procedure was repeated to ensure all TFA was removed. The residue was triturated with Et₂O to give a white solid, which was purified by preparative HPLC (gradient eluting with acetonitrile in water from 5% to 100%) to give the title compound (101 mg, 41%) as a white solid.

Analytical HPLC: purity 98.1% (System D, R_{T}=6.14 min); Analytical LCMS: purity 100% (System B, R_{T}=3.90 min), ES⁺: 455.7 [MH]⁺. HRMS calcd for C₂₅H₃₄N₄O₄: 454.2580, found 454.2586.

### Example 4

### 2-Piperazin-1-ylethyl{(1S)-1-(4-hydroxybenzyl)-1-methyl-2-[(3-methylbutyl)amino]-2-oxoethyl}carbamate dihydrochloride

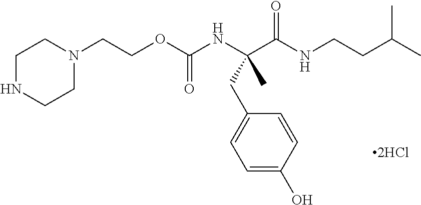

### Step 1: (S)-methyl 2-(4-hydroxybenzyl)-2-aminopropanoate hydrochloride

Thionyl chloride (5.60 mL, 76.8 mmol) was added dropwise to a stirred suspension of (S)-2-(4-hydroxybenzyl)-2-aminopropanoic acid (5.00 g, 25.6 mmol) in MeOH (100 mL) at 0° C. The reaction was allowed to warm to r.t. and left to stand for 3 weeks until the starting material was completely converted to the methyl ester. The reaction mixture was dried in vacuo to give (S)-methyl 2-(4-hydroxybenzyl)-2-aminopropanoate hydrochloride (5.13 g, 82%) as a white solid.

### Step 2: 2-(4-(tert-Butoxycarbonyl)piperazin-1-yl)ethyl(S)-2-(methoxycarbonyl)-1-(4-hydroxyphenyl)propan-2-ylcarbamate

To a stirred solution of triphosgene (594 mg, 2.0 mmol) in DCM (10 mL) at 0° C. was added a solution of tert-butyl 4-(2-hydroxyethyl)piperazine-1-carboxylate (1.38 g, 6.0 mmol) and DMAP (732 mg, 6.0 mmol) in DCM (20 mL) drop-wise over 10 minutes. The reaction mixture was stirred for 2 hours and then allowed to warm to ambient temperature. A solution of (S)-methyl 2-(4-hydroxybenzyl)-2-aminopropanoate hydrochloride (1.47 g, 6.0 mmol) and DMAP (2.12 mg, 18 mmol) in DCM (30 mL) was added over 10 minutes. The reaction mixture was stirred for 21 hours and then washed with water, 0.2M aq HCl (2×), brine, aq NaHCO₃ solution (2×) and brine. The organic layer was dried (MgSO₄), filtered and the solvent removed in vacuo. The residue was purified by reverse phase chromatography (250×26 mm column, gradient eluting with MeOH in water from 0% to 100%) and normal phase chromatography (10 g RediSep column, gradient eluting with MeOH in DCM from 0% to 5%) to give 2-(4-(tert-butoxycarbonyl)piperazin-1-yl)ethyl(S)-2-(methoxycarbonyl)-1-(4-hydroxyphenyl)propan-2-ylcarbamate (80 mg). The previous water and 0.2M HCl washes were combined, NaHCO₃ added to adjust the pH to ˜7 and then extracted with EtOAc (3×75 mL). The combined EtOAc extracts were evaporated in vacuo and the residue purified by reverse and normal phase chromatography to give a further 296 mg of the product. The total yield was of the title compound was 376 mg (14%).

### Step 3: 2-(4-(tert-Butoxycarbonyl)piperazin-1-yl)ethyl(S)-2-(carboxy)-1-(4-hydroxy-phenyl)propan-2-ylcarbamate

To a solution of 2-(4-(tert-butoxycarbonyl)piperazin-1-yl)ethyl(S)-2-(methoxycarbonyl)-1-(4-hydroxyphenyl)propan-2-ylcarbamate (376 mg, 0.81 mmol) in dioxane (20 mL) was added a solution of LiOH.H₂O (84 mg, 2.0 mmol) in water (10 mL). The reaction mixture to was stirred over the weekend. 1M HCl (2.0 mL, 2.0 mmol) was added and then concentrated in vacuo. The residue was purified by reverse phase chromatography (250×26 mm column, gradient eluting with MeOH in water from 0% to 100%). The pure fractions were combined and dried in vacuo to give 2-(4-(tert-butoxycarbonyl)piperazin-1-yl)ethyl(S)-2-(carboxy)-1-(4-hydroxyphenyl)propan-2-ylcarbamate (266 mg, 73%).

### Step 4: 2-(4-(tert-Butoxycarbonyl)piperazin-1-yl)ethyl(S)-2-(isopentylcarbamoyl)-1-(4-hydroxyphenyl)propan-2-ylcarbamate

To a solution of 2-(4-(tert-butoxycarbonyl)piperazin-1-yl)ethyl(S)-2-(carboxy)-1-(4-hydroxyphenyl)propan-2-ylcarbamate (266 mg, 0.59 mmol) in DMF (10 mL) was added HBTU (224 mg, 0.59 mmol) and DIPEA (103 μL, 0.59 mmol) followed by 3-methylbutylamine (82 μL, 0.71 mmol) and DIPEA (123 μL, 0.71 mmol). The reaction mixture was stirred overnight and then concentrated in vacuo. The residue was dissolved in EtOAc (30 mL) and washed with dilute citric acid (2×30 mL), brine (30 mL), sat aq NaHCO₃ solution (3×30 mL) and brine (30 mL). The organic layer was dried (MgSO₄) and concentrated in vacuo. The residue was purified by reverse phase chromatography and dried in vacuo to give 2-(4-(tert-butoxycarbonyl)piperazin-1-yl)ethyl(S)-2-(isopentylcarbamoyl)-1-(4-hydroxyphenyl)propan-2-ylcarbamate (217 mg, 71%) as a white foam.

### Step 5: 2-Piperazin-1-ylethyl{(1S)-1-(4-hydroxybenzyl)-1-methyl-2-[(3-methylbutyl)-amino]-2-oxoethyl}carbamate dihydrochloride

2-(4-(tert-Butoxycarbonyl)piperazin-1-yl)ethyl(S)-2-(isopentylcarbamoyl)-1-(4-hydroxy-phenyl)propan-2-ylcarbamate (217 mg, 0.42 mmol) was dissolved in DCM (10 mL) and treated with thioanisole (0.5 mL) followed by TFA (3 mL). The solution was stirred for 2 hours and then concentrated in vacuo. The residue was dissolved in 0.2M HCl in acetic acid (10 mL) and concentrated in vacuo. This procedure was repeated to ensure all the TFA was removed. The residue was triturated with Et₂O to give a white solid. This solid was purified by preparative HPLC (gradient eluting with acetonitrile in water from 5% to 100%) to give the title compound (107 mg, 52%) as a white solid.

Analytical HPLC: purity 98.9% (System D, R_{T}=6.72 min); Analytical LCMS: purity 100% (System B, R_{T}=4.18 min), ES⁺: 421.1 [MH]⁺; HRMS calcd for C₂₂H₃₆N₄O₄: 420.2737, found 420.2748.

### Example 5

### Pyridin-4-ylmethyl[(1S)-2-[benzyl)methyl)amino]-1-(4-hydroxybenzyl)-2-oxoethyl]-carbamate

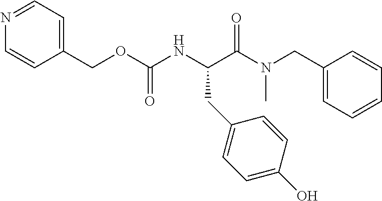

A portion of (pyridin-4-yl)methyl(S)-1-(carboxy)-2-(4-hydroxyphenyl)ethylcarbamate (Intermediate 2; 309 mg, 0.98 mmol) was dissolved in DMF (15 mL) and treated sequentially with N-methylbenzylamine (145 mg, 1.2 mmol), DIPEA (0.40 mL, 2.30 mmol) and PyBrOP (470 mg, 1.00 mmol) with stirring at 0° C. The reaction mixture was kept at 0° C. for 5 hours and then allowed to warm to r.t. overnight. The reaction mixture was concentrated in vacuo and the residue was purified by reverse phase chromatography to give pyridin-4-ylmethyl[(1S)-2-[benzyl(methyl)amino]-1-(4-hydroxybenzyl)-2-oxoethyl]carbamate (165 mg, 40%) as a white solid.

Analytical HPLC: purity 98.2% (System D, R_{T}=6.70 min); Analytical LCMS: purity 100% (System B, R_{T}=4.66 min), ES⁺: 420.1 [MH]⁺; HRMS calcd for C₂₄H₂₅N₃O₄: 419.1845, found 419.1853.

### Example 6

### Pyridin-4-ylmethyl{(1S)-1-(4-hydroxybenzyl)-2-[methyl(2-phenylethyl)amino]-2-oxo-ethyl}carbamate

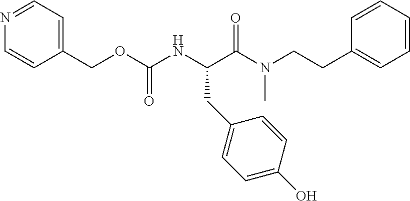

(Pyridin-4-yl)methyl(S)-1-(carboxy)-2-(4-hydroxyphenyl)ethylcarbamate (Intermediate 2; 291 mg, 0.92 mmol), N-methylphenethylamine (149 mg, 1.10 mmol), PyBrOP (457 mg, 0.98 mmol) and DIPEA (0.40 mL, 2.30 mmol) were dissolved in DMF (15 mL) cooled with an ice-water bath and stirred overnight. The reaction mixture was concentrated in vacuo. The residue was suspended in 6% aq NaHCO₃ solution (50 mL) and extracted with DCM (3×50 mL). The combined organic layers were dried (MgSO₄), filtered and concentrated in vacuo. The residue was purified by reverse phase chromatography to give pyridin-4-ylmethyl{(1S)-1-(4-hydroxybenzyl)-2-[methyl(2-phenylethyl)amino]-2-oxo-ethyl}carbamate (221 mg, 55%) as a white foam.

Analytical HPLC: purity 98.4% (System D, R_{T}=7.11 min); Analytical LCMS: purity 100% (System B, R_{T}=4.80 min), ES⁺: 434.1 [MH]⁺; HRMS calcd for C₂₅H₂₇N₃O₄: 433.2002, found 433.2012.

### Example 7

### Pyridin-4-ylmethyl{(1S)-1-(4-hydroxybenzyl)-2-[methyl(3-methylbutyl)amino]-2-oxo-ethyl}carbamate hydrochloride

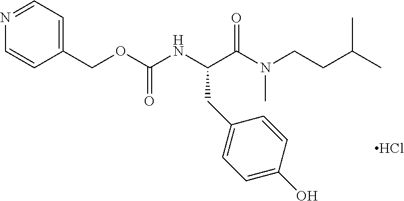

### Step 1: tert-Butyl(S)-1-(N-isopentyl-N-methylcarbamoyl)-2-(4-tert-butoxyphenyl)ethyl-carbamate

To a solution of N-(tert-butoxycarbonyl)-O-(tert-butyl)-L-tyrosine (805 mg, 2.39 mmol) in DMF (20 mL) was added N-methylisoamylamine (256 mg, 2.53 mmol) and DIPEA (0.85 mL, 4.90 mmol). The reaction mixture was cooled with an ice-water bath and PyBrOP (1.11 g, 2.40 mmol) added. The reaction mixture was stirred at 0° C. for 5 hours and then allowed to warm to r.t. overnight. The reaction mixture was concentrated in vacuo. The residue was suspended in 0.2M aq HCl (50 mL) and extracted with DCM (3×50 mL). The combined DCM extracts were dried (MgSO₄) and concentrated in vacuo to give tert-butyl(S)-1-(N-isopentyl-N-methylcarbamoyl)-2-(4-tert-butoxyphenyl)ethylcarbamate (832 mg, 83%) as a colourless gum.

### Step 2: (S)-2-Amino-3-(4-hydroxyphenyl)-N-isopentyl-N-methylpropanamide trifluoro-acetic acid

tert-Butyl(S)-1-(N-isopentyl-N-methylcarbamoyl)-2-(4-tert-butoxyphenyl)ethylcarbamate (832 mg, 1.98 mmol) was dissolved in DCM (20 mL), treated with thioanisole (1 mL) followed by TFA (5 mL), stirred overnight and then concentrated in vacuo. The residue was purified by reverse phase chromatography and dried in vacuo to give (S)-2-amino-3-(4-hydroxyphenyl)-N-isopentyl-N-methylpropanamide trifluoroacetic acid (643 mg, 86%) as a pale yellow solid.

### Step 3: Pyridin-4-ylmethyl{(1S)-1-(4-hydroxybenzyl)-2-[methyl(3-methylbutyl)amino]-2-oxoethyl}carbamate hydrochloride

4-Nitrophenyl(pyridin-4-yl)methyl carbonate (337 mg, 1.20 mmol), (S)-2-amino-3-(4-hydroxyphenyl)-N-isopentyl-N-methylpropanamide trifluoroacetic acid (359 mg, 0.95 mmol), DIPEA (0.40 mL, 2.30 mmol) and DMAP (10 mg) were dissolved in DMF (10 mL) and stirred at r.t. overnight. The reaction mixture was concentrated in vacuo. The residue was dissolved in EtOAc (50 mL) and washed with sat aq NaHCO₃ solution (5×50 mL). The organic phase was dried (MgSO₄) and concentrated in vacuo. The residue was purified by normal phase chromatography (gradient eluting with MeOH in DCM from 0% to 10%) followed by preparative HPLC (gradient eluting with acetonitrile in water from 5% to 100%) to give a white solid. The solid was dissolved in DCM (10 mL), treated with 2M HCl in Et₂O (2 mL) and dried in vacuo to give the title compound (121 mg, 29%) as a white powder.

Analytical HPLC: purity 99.4% (System D, R_{T}=6.80 min); Analytical LCMS: purity 100% (System B, R_{T}=4.36 min), ES⁺: 400.8 [MH]⁺; HRMS calcd for C₂₂H₂₉N₃O₄: 399.2158, found 399.2170.

### Example 8

### Pyridin-4-ylmethyl((1S)-3-(4-hydroxyphenyl)-1-{[methyl(2-phenylethyl)amino]-carbonyl}propyl)carbamate hydrochloride

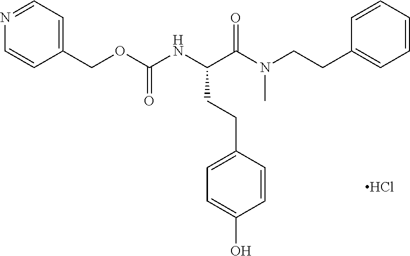

### Step 1: (Pyridin-4-yl)methyl(S)-1-(methoxycarbonyl)-3-(4-hydroxyphenyl)propyl-carbamate

Homotyrosine methyl ester hydrochloride (0.29 g, 1.10 mmol) was dissolved in DMF (10 mL) before DIPEA (0.57 mL, 3.29 mmol) and DMAP (30 mg) were added. The reaction mixture was stirred at r.t. for 5 minutes and then 4-nitrophenyl (pyridin-4-yl)methyl carbonate (316 mg, 1.15 mmol) added. The reaction mixture was stirred overnight and then concentrated in vacuo. The residue was taken up in EtOAc (30 mL) and washed with 1M aq Na₂CO₃ solution until the yellow colour of the aqueous phase had disappeared. The organic layer was dried (Na₂SO₄), filtered and evaporated to dryness. The resulting oil was purified by normal phase chromatography (10 g silica cartridge, gradient eluting with MeOH in DCM from 0% to 5%) to give (pyridin-4-yl)methyl(S)-1-(methoxycarbonyl)-3-(4-hydroxyphenyl)propylcarbamate (213 mg, 56%).

### Step 2: (Pyridin-4-yl)methyl(S)-1-(carboxy)-3-(4-hydroxyphenyl)propylcarbamate

(Pyridin-4-yl)methyl(S)-1-(methoxycarbonyl)-3-(4-hydroxyphenyl)propylcarbamate (211 mg, 0.60 mmol) was dissolved in THF (6 mL) and a 1M solution of LiOH in water (1.84 mL, 1.84 mmol) was added. The reaction mixture was stirred overnight. An aqueous solution of 1M HCl (1.84 mL, 1.84 mmol) was added and the reaction mixture was dried in vacuo to give (pyridin-4-yl)methyl(S)-1-(carboxy)-3-(4-hydroxyphenyl)propylcarbamate (137 mg, 68%).

### Step 3: Pyridin-4-ylmethyl((1S)-3-(4-hydroxyphenyl)-1-{[methyl(2-phenylethyl)amino]-carbonyl}propyl)carbamate hydrochloride

(Pyridin-4-yl)methyl(S)-1-(carboxy)-3-(4-hydroxyphenyl)propylcarbamate (137 mg, 0.41 mmol), N-methylphenethylamine (0.10 mL, 0.69 mmol) and DIPEA (0.21 mL, 1.22 mmol) were dissolved in DMF (7.5 mL) and cooled to 0° C. HCTU (268 mg, 0.64 mmol) was added and the reaction mixture stirred at 0° C. for 2 hours and then r.t for 48 hours. The reaction mixture was concentrated in vacuo. The residue was taken up in EtOAc (25 mL) and washed with 0.2M aq HCl (3×20 mL) and brine (20 mL). The organic phase was dried (Na₂SO₄), filtered and concentrated in vacuo. The residue was purified (in three iii batches) by preparative HPLC. The fractions containing product were combined and further purified by preparative HPLC. The product was dissolved in MeOH (1 mL), treated with 2M HCl in Et₂O (0.04 mL, 0.08) and concentrated in vacuo to give the title compound (35 mg, 8%) as a white solid.

Analytical HPLC: purity 99.8% (System E, R_{T}=4.59 min); Analytical LCMS: purity >99% (System B, R_{T}=4.97 min), ES⁺: 448.2 [MH]⁺; HRMS calcd for C₂₆H₂₉N₃O₄: 447.2158, found 447.2167.

### Example 9

### Pyridin-4-ylmethyl{(1S)-1-(hydroxymethyl)-2-[methyl(3-methylbutyl)amino]-2-oxo-ethyl}carbamate

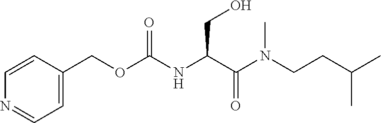

### Step 1: 9H-Fluoren-9-ylmethyl{(1S)-1-tert-butoxy-2-[methyl(3-methylbutyl)amino-2-oxo-ethyl}carbamate

To a stirred solution of 9H-fluoren-9-ylmethyl{(1S)-1-tert-butoxy-2-oxo-2-[(4-oxo-1,2,3-benzotriazin-3(4H)-yl)oxy]ethyl}carbamate (2.11 g, 4.0 mmol) in DMF (10 mL) was added N-methylisoamylamine (444 mg, 4.4 mmol). After 24 hours the solvent was removed in vacuo. The residue was taken up in EtOAc (50 mL) and washed with 5% aq citric acid solution (50 mL), sat aq NaHCO₃ solution (3×50 mL) and brine (50 mL). The EtOAc was dried (MgSO₄), filtered and concentrated in vacuo. The residue was purified by reverse phase chromatography, normal phase chromatography (35 g RediSep column, gradient eluting with MeOH in DCM from 0% to 5%) and then reverse phase chromatography. Fractions with a purity >90% by HPLC analysis were combined, evaporated and dried in vacuo to give 9H-fluoren-9-ylmethyl{(1S)-1-tert-butoxy-2-[methyl(3-methylbutyl)amino]-2-oxoethyl}carbamate (345 mg, 35%).

### Step 2: (2S)-2-Amino-2-tert-butoxy-N-methyl-N-(3-methylbutyl)acetamide

9H-fluoren-9-ylmethyl{(1S)-1-tert-butoxy-2-[methyl(3-methylbutyl)amino]-2-oxoethyl}-carbamate (345 mg, 1.4 mmol) was dissolved in piperidine (5 mL) and DMF (20 mL) and stirred overnight at r.t. The solvent mixture was removed in vacuo. The residue was purified by reverse phase chromatography and dried in vacuo to give (2S)-2-amino-2-tert-butoxy-N-methyl-N-(3-methylbutyl)acetamide (132 mg, 73%).

### Step 3: Pyridin-4-ylmethyl{(1S)-1-tert-butoxy-2-[methyl(3-methylbutypamino]-2-oxo-ethyl}carbamate

To a stirred solution of (2S)-2-amino-2-tert-butoxy-N-methyl-N-(3-methylbutyl)acetamide (122 mg, 0.50 mmol) and 4-nitrophenyl(pyridin-4-yl)methyl carbonate (137 mg, 0.50 mmol) in DMF (5 mL) was added DMAP (61 mg, 0.05 mmol). The reaction mixture was stirred at r.t. overnight and then concentrated in vacuo. The residue was taken up in EtOAc (100 mL), washed with aq sat NaHCO₃ solution (6×100 mL) and brine (50 mL), dried (MgSO₄) and concentrated in vacuo. The residue was purified by reverse phase chromatography and dried in vacuo to give pyridin-4-ylmethyl{(1S)-1-tert-butoxy-2-[methyl(3-methylbutyl)amino]-2-oxoethyl}carbamate (175 mg, 92%).

### Step 4: Pyridin-4-ylmethyl{(1S)-1-(hydroxymethyl)-2-[methyl(3-methylbutyl)amino]-2-oxoethyl}carbamate hydrochloride

To a solution of pyridin-4-ylmethyl{(1S)-1-tert-butoxy-2-[methyl(3-methylbutyl)amino]-2-oxoethyl}carbamate (175 mg, 0.46 mmol) in DCM (8 mL) was added TFA (4 mL). The reaction mixture was stirred overnight at r.t and then concentrated in vacuo. The residue was dissolved in 2M HCl in Et₂O (2.0 mL, 4.0 mmol) and acetic acid (10 mL) and then dried in vacuo. The addition of HCl and acetic acid mixture and subsequent evaporation was repeated. The residue was purified by reverse phase chromatography and then preparative HPLC. The pure fractions were combined and dried in vacuo at 45° C. for 1 week to give the title compound (70 mg, 47%) as a white crystalline solid.

Analytical HPLC: purity 99.1% (System E, R_{T}=3.71 min); Analytical LCMS: purity 100% (System B, R_{T}=4.28 min), ES⁺: 323.9 [MH]⁺.

### Example 10

### Pyridin-4-ylmethyl{(1S)-1-methyl-2-[methyl(2-phenylethyl)amino]-2-oxoethyl}-carbamate hydrochloride

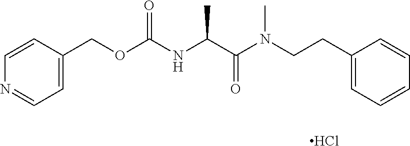

### Step 1: tert-butyl{(1S)-1-methyl-2-[methyl(2-phenylethyl)amino]-2-oxoethyl}carbamate

N-(tert-butoxycarbonyl)-L-alanine (583 mg, 3.08 mmol), N-methylphenethylamine (0.50 mL, 3.44 mmol) and DIPEA (0.60 mL, 3.45 mmol) were dissolved in DMF (25 mL) and cooled with an ice-water bath. PyBrOP (1.47 g, 3.15 mmol) was added and the reaction mixture was kept cold for five hours and then allowed to warm to r.t. overnight. The reaction mixture was concentrated in vacuo and the residue purified by reverse phase chromatography to give tert-butyl{(1S)-1-methyl-2-[methyl(2-phenylethyl)amino]-2-oxo-ethyl}carbamate (791 mg, 84%) as a colourless oil.

### Step 2: N-methyl-N-(2-phenylethyl)-L-alaninamide

A solution of tert-butyl{(1S)-1-methyl-2-[methyl(2-phenylethyl)amino]-2-oxoethyl}-carbamate (791 mg, 2.58 mmol) in DCM (20 mL) was treated with TFA (5 mL) and stirred for 2.5 hours at r.t. The reaction mixture was concentrated in vacuo. The residue was dissolved in 2M aq NaOH solution (50 mL) and extracted with DCM (3×50 mL). The combined DCM extracts were dried (MgSO₄) and concentrated in vacuo to give N-methyl-N-(2-phenylethyl)-L-alaninamide (506 mg, 95%) as a pale yellow oil.

### Step 3: Pyridin-4-ylmethyl{(1S)-1-methyl-2-[methyl(2-phenylethyl)amino]-2-oxoethyl}-carbamate hydrochloride

N-Methyl-N-(2-phenylethyl)-L-alaninamide (506 mg, 2.45 mmol) was dissolved in DMF (10 mL) and treated with DIPEA (0.50 mL, 2.88 mmol), 4-nitrophenyl(pyridin-4-yl)methyl carbonate (686 mg, 2.50 mmol) and DMAP (10 mg). The reaction mixture was stirred for four days and then concentrated in vacuo. The residue was dissolved in EtOAc (25 mL), washed with a 1M aq Na₂CO₃ solution (5×25 mL), dried (MgSO₄) and concentrated in vacuo. The residue was purified by normal phase chromatography (gradient eluting with MeOH in DCM from 0% to 5%) and then by preparative HPLC (in 3 batches) to give a colourless oil. The HCl salt was prepared by dissolving the oil in DCM (5 mL), adding 2M HCl in Et₂O (1 mL) and drying in vacuo to give the title compound (197 mg, 21%) as a white powder.

Analytical HPLC: purity 100% (System E, R_{T}=4.20 min); Analytical LCMS: purity 100% (System B, R_{T}=4.70 min), ES⁺: 341.9 [MH]⁺; HRMS calcd for C₁₉H₂₃N₃O₃: 341.1739, found 341.1754.

### Example 11

### Pyridin-4-ylmethyl{(1S)-1-benzyl-2-[methyl(2-phenylethyl)amino]-2-oxoethyl}-carbamate hydrochloride

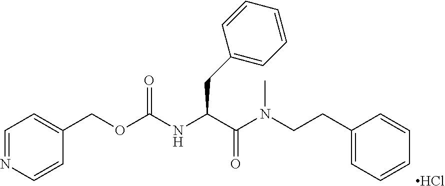

(Pyridin-4-yl)methyl(S)-1-(carboxy)-2-phenylethylcarbamate (Intermediate 3; 0.24 g, 0.80 mmol) was dissolved in DMF (8 mL) and cooled to 0° C. DIPEA (0.28 mL, 1.60 mmol) and HCTU (0.33 g, 0.80 mmol) were added followed by N-methylphenethylamine (116 μL, 0.80 mmol). The reaction mixture was allowed to warm to r.t. and stirred for 22 hours before concentrating in vacuo. The residue was taken up in EtOAc (10 mL) and washed with 1M aq citric acid solution (3×10 mL), sat aq NaHCO₃ solution (3×10 mL) and brine (10 mL). The EtOAc phase was concentrated in vacuo and the residue was purified by reverse phase chromatography. The resulting colourless oil was dissolved in DCM (5 mL) and 2M HCl in Et₂O (0.09 mL, 0.18 mmol). The solution was evaporated to dryness in vacuo to give pyridin-4-ylmethyl{(1S)-1-benzyl-2-[methyl(2-phenylethyl)amino]-2-oxoethyl}carbamate hydrochloride (0.079 g, 22%) as a hygroscopic white solid.

Analytical HPLC: purity 99.6% (System E, R_{T}=5.00 min); Analytical LCMS: purity 100% (System B, R_{T}=5.51 min), ES⁺: 418.2 [MH]⁺; HRMS calcd for C₂₅H₂₇N₃O₃: 417.2052, found 417.2058.

### Example 12

### Pyridin-4-ylmethyl[(1S)-1-benzyl-2-(dimethylamino)-2-oxoethyl]carbamate hydrochloride

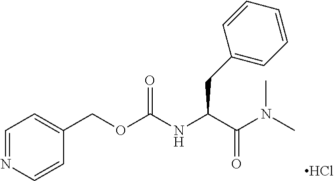

(Pyridin-4-yl)methyl(S)-1-(carboxy)-2-phenylethylcarbamate (Intermediate 3; 0.30 g, 1.0 mmol) and DIPEA (0.35 mL, 2.0 mmol) were dissolved in DMF (2 mL) and cooled to 0° C. with stirring. HCTU (0.2 M in DMF, 5.0 mL, 1.0 mmol) was added followed by the addition of dimethylamine (0.053 mL, 1.0 mmol) after 5 mins. The reaction mixture was stirred for 22 hours at r.t. before being concentrated in vacuo. The residue was dissolved in DCM (5 mL) and washed with water (5 mL) and sat aq NaHCO₃ solution (5 mL). The

DCM was removed in vacuo and the crude product was purified by normal phase column chromatography (gradient eluting with MeOH in DCM from 0% to 5%) and reverse phase chromatography. The colourless oil obtained was dissolved in acetonitrile (5 mL), and 2M HCl in Et₂O (0.25 mL, 0.50 mmol). The solution was concentrated in vacuo to give pyridin-4-ylmethyl[(1S)-1-benzyl-2-(dimethylamino)-2-oxoethyl]carbamate hydrochloride (0.161 g, 44%) as a white solid.

Analytical HPLC: purity 99.4% (System E, R_{T}=3.98 min); Analytical LCMS: purity 99% (System B, R_{T}=4.39 min), ES⁺: 328.9 [MH]⁺; HRMS calcd for C₁₈H₂₁N₃O₃: 327.1583, found 327.1593.

### Example 13

### Pyridin-4-ylmethyl{(1S)-1-benzyl-2-[(3-methylbutyl)amino]-2-oxoethyl}carbamate hydrochloride

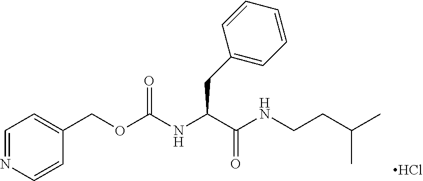

(Pyridin-4-yl)methyl(S)-1-(carboxy)-2-phenylethylcarbamate (Intermediate 3; 0.30 g, 1.0 mmol) and DIPEA (0.35 mL, 2.0 mmol) were dissolved in DMF (5 mL) and cooled to 0° C. with stirring. TBTU (0.32 g, 1.0 mmol) was added followed by the addition of isoamylamine (0.116 mL, 1.0 mmol) after 5 mins. The reaction mixture was stirred for 22 hours at r.t. before being concentrated in vacuo. The residue was dissolved in DCM (5 mL) and washed with water (5 mL) and sat aq NaHCO₃ solution (5 mL). The DCM was removed in vacuo and the crude product was purified by normal phase column chromatography (gradient eluting with MeOH in DCM from 0% to 5%) The pure fractions were combined and concentrated in vacuo. The white solid obtained was dissolved in acetonitrile (5 mL) and 2M HCl in Et₂O (0.28 mL, 0.6 mmol). The solution was concentrated in vacuo to give pyridin-4-ylmethyl{(1S)-1-benzyl-2-[(3-methylbutyl)-amino]-2-oxoethyl}carbamate hydrochloride (218 mg, 54%) as a white solid.

Analytical HPLC: purity 99.7% (System E, R_{T}=4.76 min); Analytical LCMS: purity 100% (System B, R_{T}=5.25 min), ES⁺: 370.2 [MH]⁺; HRMS calcd for C₂₁H₂₇N₃O₃: 369.2052, found 369.2062.

### Example 14

### Pyridin-4-ylmethyl{(1S)-1-benzyl-2-[isopropyl)methyl)amino]-2-oxoethyl}carbamate hydrochloride

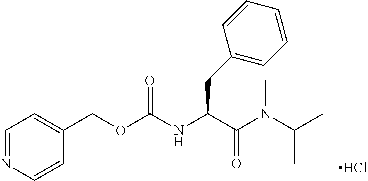

(Pyridin-4-yl)methyl(S)-1-(carboxy)-2-phenylethylcarbamate (Intermediate 3; 0.30 g, 1.0 mmol) and DIPEA (0.35 mL, 2.0 mmol) were dissolved in DMF (5 mL) and cooled to 0° C. with stirring. HCTU (0.413 g, 1.0 mmol) was added followed by the addition of N-methylisopropylamine (0.104 mL, 1.0 mmol) after 5 mins. The reaction mixture was stirred for 22 hours at r.t. before being concentrated in vacuo. The residue was dissolved in DCM (5 mL) and washed with water (5 mL) and sat aq NaHCO₃ solution (5 mL). The DCM was removed in vacuo and the residue purified by normal phase chromatography (gradient eluting with MeOH in DCM from 0% to 5%) and reverse phase chromatography. The pure fractions were combined and concentrated in vacuo. The white solid obtained was dissolved in acetonitrile (5 mL) and 2M HCl in Et₂O (0.2 mL, 0.4 mmol). The solution was concentrated in vacuo to give pyridin-4-ylmethyl{(1S)-1-benzyl-2-[isopropyl-(methyl)amino]-2-oxoethyl}carbamate hydrochloride (158 mg, 40%) as a white solid.

Analytical HPLC: purity 99.2% (System E, R_{T}=4.45 min); Analytical LCMS: purity 100% (System B, R_{T}=4.96 min), ES⁺: 356.9 [M+2H]⁺; HRMS calcd for C₂₀H₂₅N₃O₃: 355.1896, found 355.1908.

### Example 15

### Pyridin-4-ylmethyl{(1S)-1-benzyl-2-[(3,3-dimethyl-2-oxobutyl)amino]-2-oxoethyl}-carbamate hydrochloride

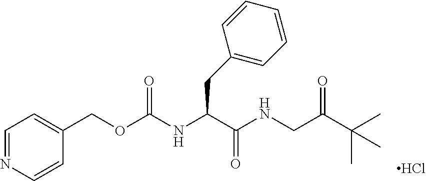

(Pyridin-4-yl)methyl(S)-1-(carboxy)-2-phenylethylcarbamate (Intermediate 3; 0.30 g, 1.0 mmol) and DIPEA (0.35 mL, 2.0 mmol) were dissolved in DMF (5 mL) and cooled to 0° C. with stirring. TBTU (0.32 g, 1.0 mmol) was added followed by the addition of 1-amino-3,3-dimethyl-butan-2-one (0.115 mg, 1.0 mmol) after 5 mins. The reaction mixture was stirred for 22 hours at r.t. before being concentrated in vacuo. The residue was dissolved in DCM (5 mL) and washed with water (5 mL) and sat aq NaHCO₃ solution (5 mL). The DCM was removed in vacuo and the crude product was purified by normal phase column chromatography (gradient eluting with MeOH in DCM from 0% to 5%) The pure fractions were combined and concentrated in vacuo. The white solid obtained was dissolved in acetonitrile (5 mL) and 2M HCl in Et₂O (0.3 ml, 0.6 mmol). The solution was concentrated in vacuo to give pyridin-4-ylmethyl{(1S)-1-benzyl-2-[(3,3-dimethyl-2-oxobutyl)amino]-2-oxoethyl}carbamate hydrochloride (162 mg, 37%) as a white solid.

Analytical HPLC: purity 99.0% (System E, R_{T}=4.51 min); Analytical LCMS: purity 99% (System B, R_{T}=5.01 min), ES⁺: 398.2 [MH]⁺; HRMS calcd for C₂₂H₂₇N₃O₄: 397.2002, found 397.2014.

### Example 16

### Pyridin-4-ylmethyl{(1S)-1-benzyl-2-[(2,2-difluoroethyl)amino]-2-oxoethyl}carbamate hydrochloride

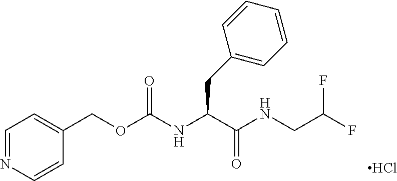

(Pyridin-4-yl)methyl(S)-1-(carboxy)-2-phenylethylcarbamate (Intermediate 3; 0.30 g, 1.0 mmol) and DIPEA (0.35 mL, 2.0 mmol) were dissolved in DMF (5 mL) and cooled to 0° C. with stirring. TBTU (0.2M in DMF, 5.0 mL, 1.0 mmol) was added followed by the addition of 2,2-difluoroethylamine (0.081 g, 1.0 mmol) after 5 mins. The reaction mixture was stirred for 22 hours at r.t. before being concentrated in vacuo. The residue was dissolved in DCM (5 mL) and washed with water (5 mL) and sat aq NaHCO₃ solution (5 mL). The DCM was removed in vacuo and the crude product was purified by normal phase column chromatography (gradient eluting with MeOH in DCM from 0% to 5%) The pure fractions were combined and concentrated in vacuo. The white solid obtained was dissolved in acetonitrile (5 mL) and 2M HCl in Et₂O (0.24 mL, 0.48 mmol). The solution was concentrated in vacuo to give pyridin-4-ylmethyl{(1S)-1-benzyl-2-[(2,2-difluoroethyl)amino]-2-oxoethyl}carbamate hydrochloride (0.192 g, 48%) as a white crystalline solid.

Analytical HPLC: purity 97.7% (System E, R_{T}=4.05 min); Analytical LCMS: purity 100% (System B, R_{T}=4.59 min), ES⁺: 364.1 [MH]⁺; HRMS calcd for C₁₈H₁₉F₂N₃O₃: 363.1394, found 363.1406.

### Example 17

### Pyridin-4-ylmethyl((1S)-1-benzyl-2-oxo-2-{[(2S)-tetrahydrofuran-2-ylmethyl]amino}-ethyl)carbamate hydrochloride

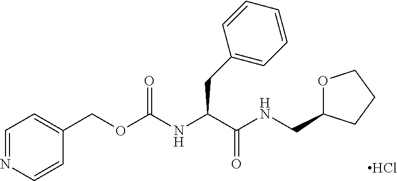

(Pyridin-4-yl)methyl(S)-1-(carboxy)-2-phenylethylcarbamate (Intermediate 3; 0.30 g, 1.0 mmol) and DIPEA (0.35 mL, 2.0 mmol) were dissolved in DMF (5 mL) and cooled to 0° C. with stirring. TBTU (0.2M in DMF, 5.0 mL, 1.0 mmol) was added followed by the addition of (S)-(+)-tetrahydrofurfurylamine (0.103 mL, 1.0 mmol) after 5 mins. The reaction mixture was stirred for 22 hours at r.t. before being concentrated in vacuo. The residue was dissolved in DCM (5 mL) and washed with water (5 mL) and sat aq NaHCO₃ solution (5 mL). The DCM was removed in vacuo and the crude product was purified by normal phase column chromatography (gradient eluting with MeOH in DCM from 0% to 5%) The pure fractions were combined and concentrated in vacuo. The white solid obtained was dissolved in acetonitrile (5 mL) and 2M HCl in Et₂O (0.12 mL, 0.24 mmol). The solution was concentrated in vacuo to give pyridin-4-ylmethyl((1S)-1-benzyl-2-oxo-2-{[(2S)-tetrahydrofuran-2-ylmethyl]amino}ethyl)carbamate hydrochloride (0.091 g, 22%) as a white crystalline solid.

Analytical HPLC: purity 100% (System E, R_{T}=3.98 min); Analytical LCMS: purity 100% (System B, R_{T}=4.47 min), ES⁺: 384.2 [MH]⁺; HRMS calcd for C₂₁H₂₅N₃O₄: 383.1845, found 383.1855.

### Example 18

### Pyridin-4-ylmethyl((1S)-1-benzyl-2-oxo-2-{[(2R)-tetrahydrofuran-2-ylmethyl]-amino}ethyl)carbamate hydrochloride

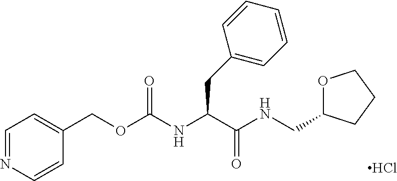

(Pyridin-4-yl)methyl(S)-1-(carboxy)-2-phenylethylcarbamate (Intermediate 3; 0.30 g, 1.0 mmol) and DIPEA (0.35 mL, 2.0 mmol) were dissolved in DMF (5 mL) and cooled to 0° C. with stirring. TBTU (0.32 g, 1.0 mmol) was added followed by the addition of (R)-(−)-tetrahydrofurfurylamine (0.103 mL, 1.0 mmol) after 5 mins. The reaction mixture was stirred for 22 hours at r.t. before being concentrated in vacuo. The residue was dissolved in DCM (5 mL) and washed with water (5 mL) and sat aq NaHCO₃ solution (5 mL). The DCM was removed in vacuo and the residue purified by normal phase chromatography (gradient eluting with MeOH in DCM from 0% to 5%) and reverse phase chromatography. The pure fractions were combined and concentrated in vacuo. The white solid obtained was dissolved in acetonitrile (5 mL) and 2M HCl in Et₂O (0.22 mL, 0.44 mmol). The solution was concentrated in vacuo to give ((1S)-1-benzyl-2-oxo-2-{[(2R)-tetrahydrofuran-2-ylmethyl]amino}ethyl)carbamate hydrochloride (175 mg, 42%) as a white solid.

Analytical HPLC: purity 99.7% (System E, R_{T}=4.01 min); Analytical LCMS: purity 99% (System B, R_{T}=4.44 min), ES⁺: 384.9 [MH]⁺; HRMS calcd for C₂₁H₂₅N₃O₄: 383.1845. found 383.1853.

### Example 19

### Pyridin-4-ylmethyl[(1S)-1-benzyl-2-morpholin-4-yl-2-oxoethyl]carbamate hydrochloride

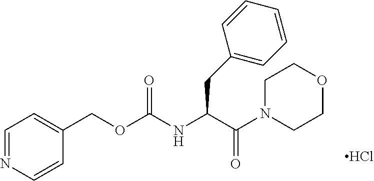

(Pyridin-4-yl)methyl(S)-1-(carboxy)-2-phenylethylcarbamate (Intermediate 3; 0.30 g, 1.0 mmol) and DIPEA (0.35 mL, 2.0 mmol) were dissolved in DMF (5 mL) and cooled to 0° C. with stirring. HCTU (0.2M in DMF, 5.0 mL, 1.0 mmol) was added followed by the addition of morpholine (0.087 mL, 1 mmol) after 5 mins. The reaction mixture was stirred for 22 hours at r.t. before being concentrated in vacuo. The residue was dissolved in DCM (5 mL) and washed with water (5 mL) and sat aq NaHCO₃ solution (5 mL). The DCM was removed in vacuo and the residue purified by normal phase chromatography (gradient eluting with MeOH in DCM from 0% to 5%) and preparative HPLC. The colourless oil obtained was dissolved in acetonitrile (5 mL) and 2M HCl in Et₂O (0.13 mL, 0.26 mmol). The solution was concentrated in vacuo to give pyridin-4-ylmethyl[(1S)-1-benzyl-2-morpholin-4-yl-2-oxoethyl]carbamate hydrochloride (0.106 g, 26%) as a white solid.

Analytical HPLC: purity 100% (System E, R_{T}=3.90 min); Analytical LCMS: purity 100% (System B, R_{T}=4.39 min), ES⁺: 370.9 [MH]⁺; HRMS calcd for C₂₀H₂₃N₃O₄: 369.1689, found 369.1704.

### Example 20

### Pyridin-4-ylmethyl{(1S)-1-(4-hydroxybenzyl)-1-methyl-2-[(3-methylbutyl)amino]-2-oxoethyl}carbamate hydrochloride

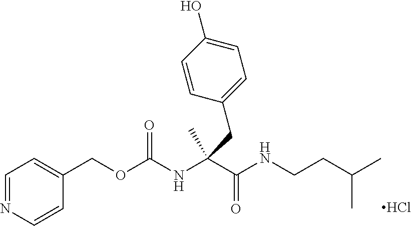

To a stirred solution of (pyridin-4-yl)methyl(S)-2-(carboxy)-1-(4-hydroxyphenyl)propan-2-ylcarbamate (Intermediate 4; 4.45 g, 13.5 mmol) in DMF (100 mL) was added solid TBTU (4.33 g, 13.5 mmol) followed by DIPEA (2.35 mL, 13.5 mmol). Once a clear solution was obtained, 3-methylbutylamine (1.88 mL, 16.2 mmol) and another portion of DIPEA (2.82 mL, 16.2 mmol) were added. After stirring overnight at ambient temperature the solvent was removed in vacuo. The residue was taken up in EtOAc (150 mL) and sequentially washed with brine (100 mL), sat aq KHCO₃ solution until the intense yellow colour had subsided (5×150 mL) and brine (100 mL). The organic layer was dried (MgSO₄), filtered and concentrated in vacuo. The desired product crystallised out from EtOAc and filtration followed by drying in vacuo gave (pyridin-4-yl)methyl(S)-2-(isopentylcarbamoyl)-1-(4-hydroxyphenyl)propan-2-ylcarbamate (4.10 g, 76%) as a white solid. The filtrate was purified by normal phase chromatography (gradient eluting with MeOH in DCM from 0% to 5%). The product was recrystallised from EtOAc and dried in vacuo to give further (pyridin-4-yl)methyl(S)-2-(isopentylcarbamoyl)-1-(4-hydroxy-phenyl)propan-2-ylcarbamate (0.85 g, 16%) as a white solid (92% combined yield).

To a vigorously stirred solution of (pyridin-4-yl)methyl(S)-2-(isopentylcarbamoyl)-1-(4-hydroxyphenyl)propan-2-ylcarbamate (10.85 g, 27 mmol) in DCM (500 mL) and MeOH (100 mL) was added 2M HCl in Et₂O (20 mL, 40 mmol, excess). The clear solution obtained was concentrated in vacuo to give pyridin-4-ylmethyl{(1S)-1-(4-hydroxybenzyl)-1-methyl-2-[(3-methylbutyl)amino]-2-oxoethyl}carbamate hydrochloride (11.9 g, quantitative) as a white foam.

Analytical HPLC: purity 100% (System E, R_{T}=4.33 min); Analytical LCMS: purity 99.7% (System B, R_{T}=4.81 min), ES⁺: 400.6 [MH]⁺; HRMS calcd for C₂₂H₂₉N₃O₄: 399.2158, found 399.2175.

### Example 21

### Pyridin-4-ylmethyl[(1S)-2-(benzylamino)-1-(4-hydroxybenzyl)-1-methyl-2-oxoethyl]-carbamate

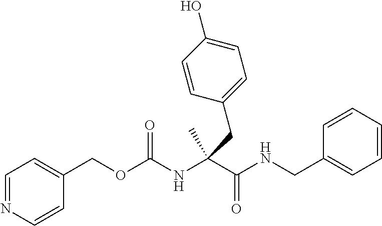

To a stirred solution of (pyridin-4-yl)methyl(S)-2-(carboxy)-1-(4-hydroxyphenyl)propan-2-ylcarbamate (Intermediate 4; 100 mg, 0.30 mmol) and DIPEA (52 μL, 0.30 mmol) in DMF (5 mL) was added solid HBTU (114 mg, 0.30 mmol) followed by benzylamine (39 μL, 0.36 mmol) and DIPEA (63 μL, 0.36 mmol). After stirring overnight at r.t. the DMF was removed in vacuo. The residue was taken up in EtOAc and washed with dilute citric acid (×2), brine, aq Na₂CO₃ solution (×2), brine. The EtOAc phase was dried (MgSO₄) and concentrated in vacuo. The residue was purified by normal phase chromatography (10 g RediSep column, gradient eluting with MeOH in DCM from 0% to 5% at 15 mL/min) and reverse phase chromatography. The pure fractions were combined and dried in vacuo to give pyridin-4-ylmethyl[(1S)-2-(benzylamino)-1-(4-hydroxybenzyl)-1-methyl-2-oxoethyl]carbamate (64 mg, 50%) as a colourless glass.

Analytical HPLC: purity 100% (System D, R_{T}=5.92 min); Analytical LCMS: purity 100% (System B, R_{T}=4.53 min), ES⁺: 420.1 [MH]⁺; HRMS calcd for C₂₄H₂₅N₃O₄: 419.1845, found 419.1846.

### Example 22

### Pyridin-4-ylmethyl((1S)-1-(4-hydroxybenzyl)-1-methyl-2-oxo-2-{[(1S)-1-phenyl-ethyl]amino}ethyl)carbamate hydrochloride

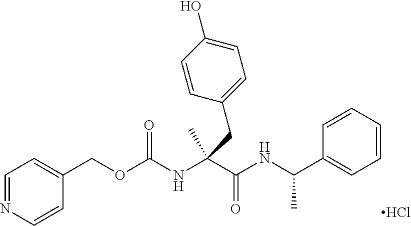

To a stirred solution of (pyridin-4-yl)methyl(S)-2-(carboxy)-1-(4-hydroxyphenyl)propan-2-ylcarbamate (Intermediate 4; 0.189 g, 0.57 mmol) in DMF (6.5 mL) was added DIPEA (0.200 mL, 1.14 mmol) followed by (S)-methylbenzylamine (0.077 mL, 0.60 mmol). The reaction mixture was cooled to 0° C. followed by the addition of HBTU (0.217 g, 0.57 mmol). The reaction was left to stir at 0° C. for 3 hours and then stirred overnight at r.t. The volatiles were removed in vacuo and the resulting residue taken up in EtOAc (30 mL) and washed with 0.2M aq HCl solution (3×20 mL) and brine (20 mL). The organic phase was dried (MgSO₄), filtered and evaporated to dryness to give a yellow oil that was purified by preparative HPLC. The product was dissolved in MeOH (1 mL) and 2M HCl in Et₂O (1 mL) was added. The clear solution obtained was concentrated in vacuo and dried in a vacuum oven at 45° C. to give pyridin-4-ylmethyl((1S)-1-(4-hydroxybenzyl)-1-methyl-2-oxo-2-{[(1S)-1-phenylethyl]amino}ethyl)carbamate hydrochloride (0.052 g, 19%) as a white solid.

Analytical HPLC: purity 100% (System E, R_{T}=4.35 min); Analytical LCMS: purity 100% (System B, R_{T}=4.79 min), ES⁺: 434.2 [MH]⁺; HRMS calcd for C₂₅H₂₇N₃O₄: 433.2002, found 433.2011.

### Example 23

### Pyridin-4-ylmethyl{(1S)-1-(4-hydroxybenzyl)-1-methyl-2-[methyl(2-phenylethyl)-amino]-2-oxoethyl}carbamate

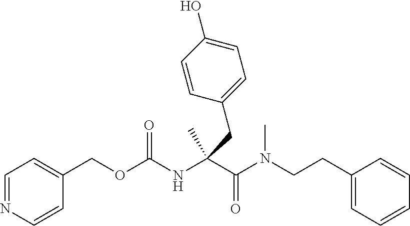

(Pyridin-4-yl)methyl(S)-2-(carboxy)-1-(4-hydroxyphenyl)propan-2-ylcarbamate (Intermediate 4; 0.24 mg, 0.72 mmol), N-methylphenethylamine (0.125 mL, 0.86 mmol) and DIPEA (0.37 mL, 2.16 mmol) were dissolved in DMF (10 mL) and cooled to 0° C. followed by the addition of PyBrOP (335 mg, 0.72 mmol). The reaction mixture was kept at 0° C. for 5 hours and left to warm to r.t. overnight. The volatiles were removed in vacuo. The yellow residue was taken up in EtOAc (30 mL) and washed with 0.5M aq HCl solution (3×20 mL) and brine (20 mL). The organic layer was dried (Na₂SO₄), filtered and concentrated in vacuo to give a yellow oil. The oil was purified by normal phase chromatography (gradient eluting with MeOH in DCM from 0% to 4%) and reverse phase chromatography to give pyridin-4-ylmethyl{(1S)-1-(4-hydroxybenzyl)-1-methyl-2-[methyl(2-phenylethyl)amino]-2-oxoethyl}carbamate (44 mg, 14%) as a white solid.

Analytical HPLC: purity 99.6% (System E, R_{T}=4.52 min); Analytical LCMS: purity 100% (System B, R_{T}=4.96 min), ES⁺: 448.1 [MH] +; HRMS calcd for C₂₆H₂₉N₃O₄: 447.2158, found 447.2164.

### Example 24

### Pyridin-4-ylmethyl{(1S)-1-benzyl-1-methyl-2-[(3-methylbutyl)amino]-2-oxoethyl}-carbamate hydrochloride

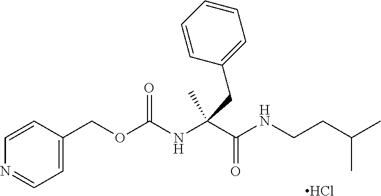

### Step 1: Methyl(2S)-2-amino-2-methyl-3-phenylpropanoate hydrochloride

To a suspension of (2S)-2-amino-2-methyl-3-phenylpropanoic acid (1.45 g, 8.1 mmol) in MeOH (50 mL) was cautiously added thionyl chloride (1.80 mL, 24.7 mmol). The reaction was stirred for 3 weeks at r.t. The reaction mixture was concentrated in vacuo to give methyl(2S)-2-amino-2-methyl-3-phenylpropanoate hydrochloride (1.86 g, 100%) as an orange brown solid.

### Step 2: (Pyridin-4-yl)methyl(S)-2-(methoxycarbonyl)-1-phenylpropan-2-ylcarbamate

Methyl(2S)-2-amino-2-methyl-3-phenylpropanoate hydrochloride (0.536 g, 2.35 mmol) and DIPEA (1.0 mL, 5.76 mmol) were dissolved in DMF (15 mL) before 4-nitrophenyl (pyridin-4-yl)methyl carbonate (0.64 g, 2.35 mmol) and DMAP (10 mg) were added. The reaction was stirred overnight at r.t. and then concentrated in vacuo. The residue was dissolved in EtOAc (25 mL) and washed with 1M aq Na₂CO₃ (5×25 mL), dried (MgSO₄), filtered and evaporated to dryness. The resulting oil was purified by normal phase chromatography (gradient eluting with MeOH in DCM from 0% to 5%) to give (pyridin-4-yl)methyl(S)-2-(methoxycarbonyl)-1-phenylpropan-2-ylcarbamate (538 mg, 1.64 mmol, 68%) as a pale yellow oil.

### Step3: (Pyridin-4-yl)methyl(S)-2-(carboxy)-1-phenylpropan-2-ylcarbamate

(Pyridin-4-yl)methyl(S)-2-(methoxycarbonyl)-1-phenylpropan-2-ylcarbamate (528 mg, 1.61 mmol) was dissolved in THF (20 mL) and a solution of LiOH.H₂O (300 mg, 7.14 mmol) in water (5 mL) was added. The reaction was left to stir overnight before adding acetic acid (1 mL). The mixture was concentrated in vacuo and the residue was purified by reverse phase chromatography to give (pyridin-4-yl)methyl(S)-2-(carboxy)-1-phenyl-propan-2-ylcarbamate (267 mg, 53%) as a white solid.

### Step 4: Pyridin-4-ylmethyl{(1S)-1-benzyl-1-methyl-2-[(3-methylbutyl)amino]-2-oxoethyl}-carbamate hydrochloride

To a stirred solution of (pyridin-4-yl)methyl(S)-2-(carboxy)-1-phenylpropan-2-yl-carbamate (267 mg, 0.85 mmol), DIPEA (0.25 mL, 1.44 mmol) and 3-methylbutylamine (0.135 mL, 1.17 mmol) in DMF (10 mL) was added solid TBTU (300 mg, 0.93 mmol). After stirring overnight at r.t. the solvent was removed in vacuo. The residue was purified by reverse phase chromatography and dried in vacuo. The residue was dissolved in Et₂O (5 mL) and treated with 2M HCl in Et₂O (1 mL) to give the title compound (275 mg, 76%) as a white solid.

Analytical HPLC: purity 99.6% (System E, R_{T}=4.85 min); Analytical LCMS: purity 100% (System B, R_{T}=5.40 min), ES⁺: 384.1 [MH]⁺; HRMS calcd for C₂₂H₂₉N₃O₃: 383.2209, found 383.2214.

### Example 25

### Pyridin-4-ylmethyl{1,1-dimethyl-2-[(3-methylbutyl)amino]-2-oxoethyl}carbamate hydrochloride

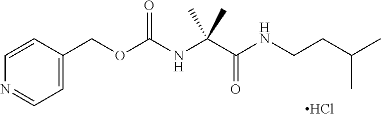

### Step 1: tert-Butyl 2-(isopentylcarbamoyl)propan-2-ylcarbamate

N-(tert-butoxycarbonyl)-2-methylalanine (1.53 g, 7.5 mmol), 3-methylbutylamine (1.0 mL, 8.6 mmol) and DIPEA (1.5 mL, 8.6 mmol) were dissolved in DMF (25 mL). TBTU (2.41 g, 7.5 mmol) was added and the reaction mixture was stirred overnight. The reaction mixture was concentrated in vacuo and the residue purified by reverse phase chromatography to give tert-butyl 2-(isopentylcarbamoyl)propan-2-ylcarbamate (1.89 g, 92%) as a white solid.

### Step 2: 2-Amino-N-isopentyl-2-methylpropanamide

To a solution of tert-Butyl 2-(isopentylcarbamoyl)propan-2-ylcarbamate (1.89 g, 6.9 mmol) in DCM (50 mL) was added TFA (10 mL) and stirred for 3 hours at r.t. The reaction mixture was concentrated in vacuo and the residue dissolved in 1M aq Na₂CO₃ solution (50 mL) and extracted with DCM (3×50 mL). The combined organic layers were dried (MgSO₄) and concentrated in vacuo to give 2-amino-N-isopentyl-2-methylpropanamide (1.06 g, 89%) as a pale orange oil.

### Step 3: Pyridin-4-ylmethyl{1,1-dimethyl-2-[(3-methylbutyl)amino]-2-oxoethyl}carbamate hydrochloride

A portion of 2-amino-N-isopentyl-2-methylpropanamide (273 mg, 1.6 mmol) was dissolved in DMF (5 mL) and treated with DIPEA (0.35 mL, 2.0 mmol), 4-nitrophenyl (pyridin-4-yl)methyl carbonate (493 mg, 1.8 mmol) and DMAP (10 mg). The reaction mixture was stirred for three days before being concentrated in vacuo. The residue was dissolved in EtOAc (25 mL), washed with a 1M aq Na₂CO₃ solution (5×25 mL), dried (MgSO₄) and concentrated in vacuo. The residue was purified by normal phase chromatography (gradient eluting with MeOH in DCM from 0% to 5%) to give a colourless oil. This oil was dissolved in DCM (5 mL), treated with 2M HCl in Et₂O (1 mL) and concentrated in vacuo to give the title compound (307 mg, 56%) as a white powder.

Analytical HPLC: purity 99.3% (System E, R_{T}=3.86 min); Analytical LCMS: purity 98.5% (System B, R_{T}=4.32 min), ES⁺: 308.0 [MH]⁺; HRMS calcd for C₁₆H₂₅N₃O₃: 307.1896, found 307.1897.

### Example 26

### (2,6-Dimethylpyridin-4-yl)methyl{1,1-dimethyl-2-[(3-methylbutyl)amino]-2-oxo-ethyl}carbamate hydrochloride

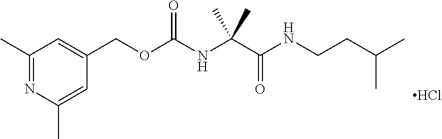

To a stirred solution of 2,6-dimethylpyridin-4-yl)methyl 2-(carboxy)propan-2-ylcarbamate (Intermediate 5; 300 mg, 1.0 mmol) and DIPEA (0.52 mL, 3.0 mmol) in DMF (5 mL) at 0° C. were added isoamylamine (0.116 mL, 1.0 mmol) and solid TBTU (321 mg, 1.0 mmol). After stirring overnight at r.t. the DMF was removed in vacuo. The residue was dissolved in DCM (5 mL) and washed with water (5 mL) and sat aq NaHCO₃ solution (5 mL). The organic phase was concentrated in vacuo and the residue purified by normal phase chromatography (gradient eluting with MeOH in DCM from 0% to 2%) and preparative HPLC. The pure fractions were combined and concentrated in vacuo. The white solid obtained was dissolved in MeOH (3 mL), 2M HCl in Et₂O (0.25 mL, 0.5 mmol) added and the solution concentrated in vacuo to give (2,6-dimethylpyridin-4-yl)methyl{1,1-dimethyl-2-[(3-methylbutyl)amino]-2-oxoethyl}carbamate hydrochloride (63 mg, 17%) as a white solid.

Analytical HPLC: purity 99.8% (System E, R_{T}=4.06 min); Analytical LCMS: purity 100% (System C, R_{T}=5.65 min), ES⁺: 336.5 [MH]⁺; HRMS calcd for C₁₈H₂₉N₃O₃: 355.2209, found 355.2212.

### Example 27

### (2,6-Dimethylpyridin-4-yl)methyl{1,1-dimethyl-2-[methyl(3-methylbutyl)amino]-2-oxoethyl}carbamate hydrochloride

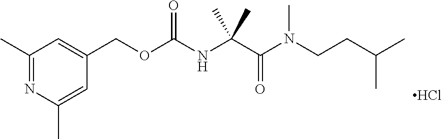

To a stirred solution 2,6-dimethylpyridin-4-yl)methyl 2-(carboxy)propan-2-ylcarbamate (Intermediate 5; 300 mg, 1.1 mmol) and DIPEA (0.52 mL, 3.0 mmol) in DMF (5 mL) at 0° C. was added N-methylisoamylamine (101 mg, 1.0 mmol) and solid HBTU (379 mg, 1.0 mmol). After stirring overnight at ambient temperature the DMF was removed in vacuo. The residue was dissolved in DCM (5 mL) and washed with water (5 mL) and sat aq NaHCO₃ solution (5 mL). The organic phase was concentrated in vacuo and the residue was purified by normal phase chromatography (gradient eluting with MeOH in DCM from 0% to 2%) abd preparative HPLC. The colourless oil obtained was dissolved in DCM (3 mL), 2M HCl in Et₂O (0.5 mL, 1.0 mmol) was added and the solution concentrated in vacuo to give (2,6-dimethylpyridin-4-yl)methyl{1,1-dimethyl-2-[methyl(3-methylbutyl)-amino]-2-oxoethyl}carbamate hydrochloride (129 mg, 33%) as a white solid.

Analytical HPLC: purity 100% (System E, R_{T}=4.26 min); Analytical LCMS: purity 97.9% (System C, R_{T}=5.80 min), ES⁺: 350.5 [MH] +; HRMS calcd for C₁₉H₃₁N₃O₃: 349.2365, found 349.2364.

### Example 28

### (2,6-Dimethylpyridin-4-yl)methyl(1,1-dimethyl-2-morpholin-4-yl-2-oxoethyl)-carbamate hydrochloride

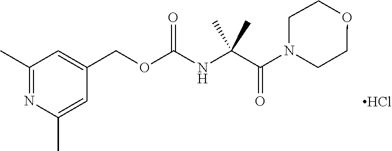

To a stirred solution of 2,6-dimethylpyridin-4-yl)methyl 2-(carboxy)propan-2-ylcarbamate (Intermediate 5; 300 mg, 1.1 mmol) and DIPEA (0.52 mL, 3.0 mmol) in DMF (5 mL) at 0° C. was added morpholine (0.087 mL, 1.0 mmol) and solid HBTU (379 mg, 1.0 mmol). After stirring overnight at r.t. the DMF was removed in vacuo. The residue was dissolved in DCM (5 mL) and washed with water (5 mL) and sat aq NaHCO₃ solution (5 mL). The organic phase was concentrated in vacuo and the residue purified by normal phase chromatography (gradient eluting with MeOH in DCM from 0% to 5%) and preparative HPLC. The colourless oil obtained was dissolved in MeOH (3 mL), 2M HCl in Et₂O (0.25 mL, 0.5 mmol) added and the solution concentrated in vacuo to (2,6-dimethylpyridin-4-yl)methyl (1,1-dimethyl-2-morpholin-4-yl-2-oxoethyl)carbamate hydrochloride (21 mg, 6%) as a white solid.

Analytical HPLC: purity 99.8% (System E, R_{T}=3.09 min); Analytical LCMS: purity 100% (System C, R_{T}=4.65 min), ES⁺: 336.4 [MH]⁺; HRMS calcd for C₁₇H₂₅N₃O₄: 335.1845, found 335.1854.

### Example 29

### (2,6-Dimethylpyridin-4-yl)methyl{2-[(2R,6S)-2,6-dimethylmorpholin-4-yl]-1,1-dimethyl-2-oxoethyl}carbamate

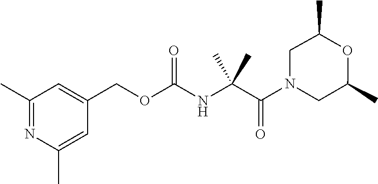

To a stirred solution of 2,6-dimethylpyridin-4-yl)methyl 2-(carboxy)propan-2-ylcarbamate (Intermediate 5; 604 mg, 2.0 mmol) and DIPEA (1.0 mL, 6.0 mmol) in DMF (20 mL) at 0° C. was added cis-2,6-dimethylmorpholine (0.246 mL, 2.0 mmol) and solid HCTU (827 mg, 2.0 mmol). After stirring overnight at r.t. the DMF was removed in vacuo and the residue purified by normal phase chromatography (gradient eluting with MeOH in DCM from 0% to 5%) and reverse phase chromatography. The pure fractions were combined and concentrated in vacuo to give (2,6-dimethylpyridin-4-yl)methyl{2-[(2R,6S)-2,6-dimethyl-morpholin-4-yl]-1,1-dimethyl-2-oxoethyl}carbamate (174 mg, 24%) as a white solid.

Analytical HPLC: purity 99.5% (System E, R_{T}=3.47 min); Analytical LCMS: purity 100% (System C, R_{T}=5.08 min), ES⁺: 364.5 [MH]⁺; HRMS calcd for C₁₉H₂₉N₃O₄: 363.2158, found 363.2169

### Example 30

### (2,6-Dimethylpyridin-4-yl)methyl{(1S)-1-(4-hydroxybenzyl)-1-methyl-2-[(3-methyl-butyl)amino]-2-oxoethyl}carbamate hydrochloride

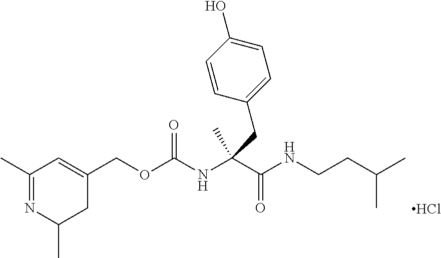

To a stirred solution of (2,6-dimethylpyridin-4-yl)methyl(S)-2-(carboxy)-1-(4-hydroxy-phenyl)propan-2-ylcarbamate (Intermediate 6; 358 mg, 1.0 mmol) and DIPEA (0.348 ml, 2.0 mmol) in DMF (5 mL) at 0° C. was added isoamylamine (0.232 mL, 2.0 mmol) and solid TBTU (321 mg, 1.0 mmol). After stirring overnight at r.t the DMF was removed in vacuo. The residue was dissolved in DCM (7 mL) and washed with water (5 mL) and sat aq NaHCO₃ solution (5 mL). The DCM was removed in vacuo and the residue purified by normal phase chromatography (gradient eluting with MeOH in DCM from 0% to 5%) and reverse phase chromatography. The pale yellow solid obtained was dissolved in MeOH (4 mL), 2M HCl in Et₂O (0.7 mL, 1.4 mmol) added and the solution concentrated in vacuo to give (2,6-dimethylpyridin-4-yl)methyl{(1S)-1-(4-hydroxybenzyl)-1-methyl-2-[(3-methyl-butyl)amino]-2-oxoethyl}carbamate hydrochloride (281 mg, 61%) as a white solid.

Analytical HPLC: purity 99.8% (System E, R_{T}=4.49 min); Analytical LCMS: purity 100% (System C, R_{T}=6.04 min), ES⁺: 428.5 [MH]⁺; HRMS calcd for C₂₄H₃₃N₃O₄: 427.2471, found 427.2489.

### Example 31

### (2,6-Dimethylpyridin-4-yl)methyl[(1S)-1-(4-hydroxybenzyl)-1-methyl-2-morpholino-4-yl-2-oxoethyl]carbamate hydrochloride

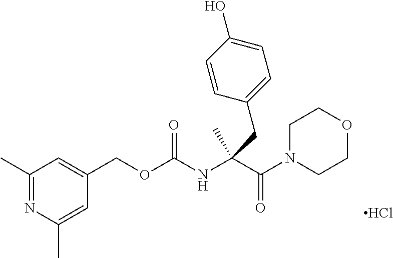

To a stirred solution of (2,6-dimethylpyridin-4-yl)methyl(S)-2-(carboxy)-1-(4-hydroxyphenyl)propan-2-ylcarbamate (Intermediate 6; 358 mg, 1.0 mmol) and DIPEA (0.348 mL, 1.0 mmol) in DMF (5 mL) at 0° C. was added morpholine (0.174 mL, 2.0 mmol) and solid HBTU (379 mg, 1.0 mmol). After stirring overnight at r.t. the DMF was removed in vacuo. The residue was dissolved in DCM (7 mL) and washed with water (5 mL) and sat aq NaHCO₃ solution (5 mL). The DCM was removed in vacuo and the residue purified by normal phase chromatography (gradient eluting with MeOH in DCM from 0% to 5%) and reverse phase chromatography. The white solid obtained was dissolved in MeOH (3 mL), 2M HCl in Et₂O (0.4 mL, 0.8 mmol) added and the solution concentrated in vacuo to give (2,6-dimethylpyridin-4-yl)methyl[(1S)-1-(4-hydroxybenzyl)-1-methyl-2-morpholino-4-yl-2-oxoethyl]carbamate hydrochloride (173 mg, 37%) as a white solid.

Analytical HPLC: purity 99.7% (System E, R_{T}=3.54 min); Analytical LCMS: purity 100% (System C, R_{T}=5.11 min), ES⁺: 428.5 [MH]⁺; HRMS calcd for C₂₃H₂₉N₃O₅: 427.2107, found 427.2118.

### Example 32

### (Pyridin-4-yl)methyl(S)-2-(isopentylcarbamoyl)-1-(3,5-ditritium-4-hydroxyphenyl)-propan-2-ylcarbamate trifluoroacetate.

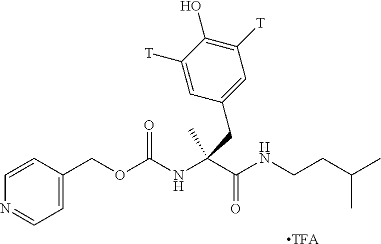

Step 1: (S)-2-(4-Hydroxybenzyl)-2-amino-N-isopentylpropanamide

A suspension of (pyridin-4-yl)methyl(S)-2-(isopentylcarbamoyl)-1-(4-hydroxyphenyl)-propan-2-ylcarbamate hydrochloride (Example 20; 0.87 g, 2.0 mmol) in MeOH (8 mL) was purged with argon. Palladium black (catalytic amount) was added and the system purged with argon before adding 1,4-cyclohexadiene (1.9 mL, 20 mmol). The reaction was stirred at 25-30° C. for 2 h, using a warm water bath. The reaction mixture was filtered through Celite® and the residue washed with MeOH (50 mL). The combined filtrates were evaporated in vacuo to give a light yellow oil which was purified by reverse phase chromatography. The pure factions were combined and concentrated in vacuo to give (S)-2-(4-hydroxybenzyl)-2-amino-N-isopentylpropanamide (360 mg, 68%) as a colourless oil.

### Step 2: (S)-2-(4-Hydroxy-3,5-diiodobenzyl)-2-amino-N-isopentylpropanamide

(S)-2-(4-Hydroxybenzyl)-2-amino-N-isopentylpropanamide (0.150 g, 0.57 mmol) was dissolved in acetonitrile (10 mL) and NaI (0.17 g, 1.14 mmol) was added and the reaction mixture was purged with argon three times. The reaction mixture was cooled to 0° C. and a solution of chloramines T (0.26 g, 1.14 mmol) in acetonitrile (15 mL) was added. The reaction was stirred at 0° C. for 20 minutes and then allowed to warm to r.t. overnight. The solvent was removed in vacuo and the residue dissolved in EtOAc (40 mL) and washed with 10% aqueous Na₂S₂O₃ solution (3×30 mL). The organic phase was dried (MgSO4), filtered and concentrated in vacuo to give a residue that was purified by reverse phase chromatography to give (S)-2-(4-hydroxy-3,5-diiodobenzyl)-2-amino-N-isopentyl-propanamide (48 mg, 16%) as a white solid.

### Step 3: (S)-2-(3,5-Ditritium-4-hydroxy-benzyl)-2-amino-N-isopentylpropanamide

A solution of (S)-2-(4-hydroxy-3,5-diiodobenzyl)-2-amino-N-isopentylpropanamide (21.1 mg, 0.04 mmol), 10% Palladium on carbon (17 mg) and DIPEA (0.1 mL) in DMAP (1.4 mL) were stirred under 10 Ci tritium gas for 2 hours. The solution was filtered, evaporated to dryness, and labile tritium removed by repeated evaporations to dryness from ethanol. Yield=2.3 Ci. Analysis by TLC (silica, DCM:MeOH:ammonia (90:10:1)) showed a single major product corresponding to (S)-2-(3,5-ditritium-4-hydroxy-benzyl)-2-amino-N-isopentylpropanamide, so material was used directly without purification in the next stage.

### Step 4: (Pyridin-4-yl)methyl(S)-2-(isopentylcarbamoyl)-1-(3,5-ditritium-4-hydroxy-phenyl)propan-2-ylcarbamate trifluoroacetate

(S)-2-(3,5-Ditritium-4-hydroxy-benzyl)-2-amino-N-isopentylpropanamide (1.15 Ci) was evaporated to dryness and dissolved in DMF (0.75 mL) containing K₂CO₃ (3.28 mg). This was stirred at r.t. under nitrogen and 4-nitrophenyl (pyridin-4-yl)methyl carbonate (5.87 mg, 0.02 mmol) was added. Stirring was continued at r.t. under nitrogen for 2.5 hours. TLC analysis was inconclusive so the reaction was worked up at this stage by evaporation to dryness and redissolving in water:acetonitrile:TFA for HPLC purification. The material was purified by reverse-phase HPLC using a water:acetonitrile:TFA gradient system. The title compound was collected, evaporated to dryness, and redissolved in ethanol.

Analysis of (pyridin-4-yl)methyl(S)-2-(isopentylcarbamoyl)-1-(3,5-ditritium-4-hydroxy-phenyl)propan-2-ylcarbamate trifluoracetate:
LC-MS: 404.4 [MH⁺]
Specific activity determined by MS: 1.78 TBq/mmol (48 Ci/mmol)
MW at this specific activity: 403 g/mol
Radioactive concentration: 74.0 MBq/ml (2 mCi/ml)
Radiochemical purity by HPLC: 98.3%

Biological Test

Measurement of Overnight Body Weight Change in Male C57 b1/6 mice

This model studies the effects of compounds on body weight gain during the pm-am period in order to maximise the effective window. Typically the mice gain about 1 g in weight during the dark phase and then loose the majority of this weight gain during the light phase, as represented in FIG. 1. The weight difference over any 24 hour period is very small whilst the weight difference between the beginning of the dark phase and the beginning of the light phase (pm-am) is maximal.

It is important to measure body weight change over the dark phase. If mice are dosed with an active compound on two consecutive days and the bodyweight change is recorded 48 hours after the first dose then no significant effect is observed. However if the body weight change over the dark phase only is considered a significant and robust effect is seen. This is because the mice rebound during the light phase to compensate for the lack of weight gain over the dark phase. Very active long lasting compounds may also diminish this rebound and reduce the body weight over the 48 hours.

Weight Change Over Consecutive Days in C57b1/6 Male Mice:

The weight difference between the beginning of the dark phase and the beginning of the light phase (pm-am) is greater than the weight difference measured between pm and pm on 2 consecutive days. The effect of the compounds on the pm-am difference was therefore studied in order to maximise the effect window.

C57 b1/6 mice were grouped (5 per cage) and left 5 days for acclimatisation. A single intraperitoneally (ip) administered dose (60 mg/kg) was given just prior to the dark phase. Compounds were either water soluble or dissolved in up to 3% cremophor (in this case the vehicle also contained cremophor). The pH was adjusted from a minimum of 5.5 to a maximum of 8 depending on the nature of the compound.

As shown in FIGS. 2 and 3, compounds of Formula (I) are useful for decreasing body weight in mice.

Leptin Assay in Non-Recombinant System

Although well-characterised in recombinant systems (e.g. ObRb-transfected HEK293 cells), where leptin elicits a very marked increase in STAT3 phosphorylation, these systems have often failed to provide an accurate measure of activity of a test compound towards the leptin receptor. It seems that overexpression of the receptor (as well as the possibility for different drugs to act on different parts of the signaling pathway triggered by leptin association with its receptor) results in most cases in the absence of activity of the drugs tested.

The leptin receptor expression in non-recombinant system is often fluctuating and care must be given to identify a system where signal stability remains within experiments. Using such a system, leptin receptor antagonist mimetics could be identified by evaluating their action vs. leptin (see below).

Leptin is produced chiefly in adipose cells, but in humans, mRNA encoding leptin is also present in the placenta. Here, leptin might play an important proliferative role in the microvasculature. The possibility to use this hypothesis in a native cell line was evaluated.

JEG-3 Protocol

In JEG-3 cells (choriocarcinoma cell line) leptin is able to stimulate proliferation up to 3 fold (Biol. Reprod. (2007) 76: 203-10). Leptin also causes a concentration-dependent increase in [³H]-thymidine incorporation in JEG-3 cells (FIG. 4, maximal effect at 100 nM (EC₅₀=2.1 nM)). The radioactivity incorporated by the cells is an index of their proliferative activity and is measured in counts per minute (CPM) with a liquid scintillation beta counter.

This finding can be applied to test whether a compound is able to either reproduce the effect of leptin on cell proliferation (leptin receptor agonist mimetic) (i.e., a given compound will cause an increase in incorporated [³H]-Thymidine by the cells) or to inhibit the effect of leptin (antagonistic effect) by preventing the leptin-mediated increase in [³H]-thymidine incorporation.

This approach has the advantage of using a non-recombinant system and has reasonable reproducibility and robustness.

Measurement of Brain Penetration

The test species (rodent) is given a bolus dose of the substrate under investigation, usually via intravenous (IV) or oral (PO) routes. At appropriate time points, blood samples are taken and the resultant plasma extracted and analysed for substrate concentration and, where appropriate, metabolite concentration. At similar time points, animals from another group are sacrificed, brains isolated and the brain surface cleaned. Brain samples are then homogenised, extracted and analysed for substrate concentration and, where appropriate, metabolite concentration. Alternatively, microdialysis probes are implanted into one or more brain regions of the test species and samples collected at appropriate time points for subsequent analysis. This method has the advantage of measuring only extra-cellular substrate concentration. Plasma and brain concentrations are then compared and ratios calculated, either by comparison of averaged concentrations at individual time points, or by calculation of the area-under-the-curve (AUC) of the concentration-time plots.

1. A compound of formula (I) 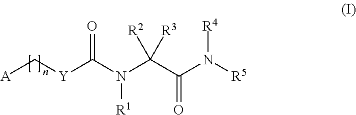
or a pharmaceutically acceptable salt, solvate, hydrate, geometrical isomer, tautomer, optical isomer or N-oxide thereof, wherein:
A is selected from pyridinyl and piperazinyl, each of which is optionally substituted with one or more C₁₋₄-alkyl groups;
Y is selected from O, N(R⁶) and CH₂;
R¹ is selected from hydrogen and C₁₋₄-alkyl;
R² is selected from hydrogen and C₁₋₄-alkyl;
R³ is selected from C₁₋₄-alkyl, hydroxy-C₁₋₄-alkyl and phenyl-C₁₋₄-alkyl, wherein phenyl is optionally substituted with one or more substituents independently selected from halogen, hydroxy, cyano, CF₃, C₁₋₄-alkyl and C₁₋₄-alkoxy;
R⁴ is selected from hydrogen and C₁₋₄-alkyl;
R⁵ is selected from C₁₋₆-alkyl (optionally substituted with one or more substituents independently selected from oxo and fluoro), phenyl-C₁₋₆-alkyl (wherein phenyl is optionally substituted with one or more substituents independently selected from halogen, hydroxy, cyano, CF₃, C₁₋₆-alkyl and C₁₋₆-alkoxy) and heterocyclyl-C₁₋₆-alkyl; or
R⁴ and R⁵, together with the nitrogen atom to which they are bound, form a saturated heterocyclic ring which is optionally substituted with one or more C₁₋₄-alkyl groups;
R⁶ is selected from hydrogen and C₁₋₄-alkyl; and
n is 1, 2 or 3;
with the proviso that the compound is not selected from:
N,3-dimethyl-2-[[[methyl(2-pyridinylmethyl)amino]carbonyl]amino]butanamide; and
N-[(1S)-1-[[[(1S)-1-(1,3-dioxolan-2-yl)-3-methylbutyl]amino]carbonyl]-2-methylpropyl]-3-pyridinepropanamide.

2. A compound according to claim 1, wherein Y is O.

3. A compound according to claim 1 or 2, wherein n is 1 or 2.

4. A compound according to claim 1, which is selected from:
2-piperazin-1-ylethyl{(1S)-1-(4-hydroxybenzyl)-2-[methyl(3-methylbutyl)-amino]-2-oxoethyl}carbamate;
2-piperazin-1-ylethyl[(1S)-2-[benzyl(methyl)amino]-1-(4-hydroxybenzyl)-2-oxo-ethyl]carbamate;
2-piperazin-1-ylethyl{(1S)-1-(4-hydroxybenzyl)-2-[methyl(2-phenylethyl)-amino]-2-oxoethyl}carbamate;
2-piperazin-1-ylethyl{(1S)-1-(4-hydroxybenzyl)-1-methyl-2-[(3-methylbutyl)-amino]-2-oxoethyl}carbamate;
pyridin-4-ylmethyl[(1S)-2-[benzyl(methyl)amino]-1-(4-hydroxybenzyl)-2-oxo-ethyl]carbamate;
pyridin-4-ylmethyl{(1S)-1-(4-hydroxybenzyl)-2-[methyl(2-phenylethyl)amino]-2-oxoethyl}carbamate;
pyridin-4-ylmethyl{(1S)-1-(4-hydroxybenzyl)-2-[methyl(3-methylbutyl)amino]-2-oxoethyl}carbamate;
pyridin-4-ylmethyl((1S)-3-(4-hydroxyphenyl)-1-{[methyl(2-phenylethyl)amino]-carbonyl}propyl)carbamate;
pyridin-4-ylmethyl{(1S)-1-(hydroxymethyl)-2-[methyl(3-methylbutyl)amino]-2-oxoethyl}carbamate;
pyridin-4-ylmethyl{(1S)-1-methyl-2-[methyl(2-phenylethyl)amino]-2-oxoethyl}-carbamate;
pyridin-4-ylmethyl{(1S)-1-benzyl-2-[methyl(2-phenylethyl)amino]-2-oxoethyl}-carbamate;
pyridin-4-ylmethyl[(1S)-1-benzyl-2-(dimethylamino)-2-oxoethyl]carbamate;
pyridin-4-ylmethyl{(1S)-1-benzyl-2-[(3-methylbutyl)amino]-2-oxoethyl}-carbamate;
pyridin-4-ylmethyl{(1S)-1-benzyl-2-[isopropyl(methyl)amino]-2-oxoethyl}-carbamate;
pyridin-4-ylmethyl{(1S)-1-benzyl-2-[(3,3-dimethyl-2-oxobutyl)amino]-2-oxo-ethyl}carbamate;
pyridin-4-ylmethyl{(1S)-1-benzyl-2-[(2,2-difluoroethyl)amino]-2-oxoethyl}-carbamate;
pyridin-4-ylmethyl((1S)-1-benzyl-2-oxo-2-{[(2S)-tetrahydrofuran-2-ylmethyl]-amino}ethyl)carbamate;
pyridin-4-ylmethyl((1S)-1-benzyl-2-oxo-2-{[(2R)-tetrahydro furan-2-ylmethyl]-amino}ethyl)carbamate;
pyridin-4-ylmethyl[(1S)-1-benzyl-2-morpholin-4-yl-2-oxoethyl]carbamate;
pyridin-4-ylmethyl{(1S)-1-(4-hydroxybenzyl)-1-methyl-2-[(3-methylbutyl)-amino]-2-oxoethyl}carbamate;
pyridin-4-ylmethyl[(1S)-2-(benzylamino)-1-(4-hydroxybenzyl)-1-methyl-2-oxo-ethyl]carbamate;
pyridin-4-ylmethyl((1S)-1-(4-hydroxybenzyl)-1-methyl-2-oxo-2-{[(1S)-1-phenyl-ethyl]amino}ethyl)carbamate;
pyridin-4-ylmethyl{(1S)-1-(4-hydroxybenzyl)-1-methyl-2-[methyl(2-phenyl-ethyl)amino]-2-oxoethyl}carbamate;
pyridin-4-ylmethyl{(1S)-1-benzyl-1-methyl-2-[(3-methylbutyl)amino]-2-oxo-ethyl}carbamate;
pyridin-4-ylmethyl{1,1-dimethyl-2-[(3-methylbutyl)amino]-2-oxoethyl}-carbamate;
(2,6-dimethylpyridin-4-yl)methyl{1,1-dimethyl-2-[(3-methylbutyl)amino]-2-oxo-ethyl}carbamate;
(2,6-dimethylpyridin-4-yl)methyl{1,1-dimethyl-2-[methyl(3-methylbutyl)amino]-2-oxoethyl}carbamate;
(2,6-dimethylpyridin-4-yl)methyl(1,1-dimethyl-2-morpholin-4-yl-2-oxoethyl)-carbamate;
(2,6-dimethylpyridin-4-yl)methyl{2-[(2R,6S)-2,6-dimethylmorpholin-4-yl]-1,1-dimethyl-2-oxoethyl}carbamate;
(2,6-dimethylpyridin-4-yl)methyl{(1S)-1-(4-hydroxybenzyl)-1-methyl-2-[(3-methylbutyl)amino]-2-oxoethyl}carbamate; and
(2,6-dimethylpyridin-4-yl)methyl[(1S)-1-(4-hydroxybenzyl)-1-methyl-2-morpholino-4-yl-2-oxoethyl]carbamate.

5. A pharmaceutical formulation containing a compound according to any one of claims 1 to 4 as active ingredient, in combination with a pharmaceutically acceptable diluent or carrier.

6. A compound according to any one of claims 1 to 4 for use in therapy.

7. A compound according to any one of claims 1 to 4 for use in the treatment or prevention of conditions or diseases associated with weight gain.

8. The compound according to claim 7, wherein the condition or disease is obesity, type 2 diabetes, lipodystrophy, insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain or macular degeneration.

9. A compound according to any one of claims 1 to 4 for use in the treatment or prevention of severe weight loss, dysmenorrhea, amenorrhea, female infertility or immunodeficiency, or in the treatment of wound healing.

10. A compound according to any one of claims 1 to 4 for use in the treatment or prevention of inflammatory conditions or diseases, low level inflammation associated with obesity and excess plasma leptin, atherosclerosis, macro or micro vascular complications of type 1 or 2 diabetes, retinopathy, nephropathy, autonomic neuropathy, or blood vessel damage caused by ischaemia or atherosclerosis.

11. A compound according to any one of claims 1 to 4 for use in the inhibition of angiogenesis.

12. Use of a compound according to any one of claims 1 to 4 in the manufacture of a medicament for the treatment or prevention of conditions or diseases associated with weight gain.

13. The use according to claim 12, wherein the condition or disease is obesity, type 2 diabetes, lipodystrophy, insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain or macular degeneration.

14. Use of a compound according to any one of claims 1 to 4 in the manufacture of a medicament for the treatment or prevention of severe weight loss, dysmenorrhea, amenorrhea, female infertility or immunodeficiency, or for the treatment of wound healing.

15. Use of a compound according to any one of claims 1 to 4 in the manufacture of a medicament for the treatment or prevention of inflammatory conditions or diseases, low level inflammation associated with obesity and excess plasma leptin, atherosclerosis, macro or micro vascular complications of type 1 or 2 diabetes, retinopathy, nephropathy, autonomic neuropathy, or blood vessel damage caused by ischaemia or atherosclerosis.

16. Use of a compound according to any one of claims 1 to 4 in the manufacture of a medicament for the inhibition of angiogenesis.

17. A method for treatment or prevention of conditions or diseases associated with weight gain, which comprises administering to a mammal, including man, in need of such treatment an effective amount of a compound according to any one of claims 1 to 4.

18. The method according to claim 17, wherein the condition or disease is obesity, type 2 diabetes, lipodystrophy, insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain or macular degeneration.

19. A method for treatment or prevention of severe weight loss, dysmenorrhea, amenorrhea, female infertility or immunodeficiency, or for treatment of wound healing, which comprises administering to a mammal, including man, in need of such treatment an effective amount of a compound according to any one of claims 1 to 4.

20. A method for treatment or prevention of inflammatory conditions or diseases, low level inflammation associated with obesity and excess plasma leptin, atherosclerosis, macro or micro vascular complications of type 1 or 2 diabetes, retinopathy, nephropathy, autonomic neuropathy, or blood vessel damage caused by ischaemia or atherosclerosis, which comprises administering to a mammal, including man, in need of such treatment an effective amount of a compound according to any one of claims 1 to 4.

21. A method for inhibition of angiogenesis, which comprises administering to a mammal, including man, in need of such treatment an effective amount of a compound according to any one of claims 1 to 4.
